(19) United States
(12) Patent Application Publication
(10) Pub. No.: US 2023/0408438 A1
(43) Pub. Date: Dec. 21, 2023

(54) ELECTROCATALYTIC POLYMER DEVICE FOR BIOLOGICAL DETECTION

(71) Applicant: Oregon State University, Corvallis, OR (US)

(72) Inventors: Li-Jing Cheng, Corvallis, OR (US); Sanjida Yeasmin, Corvallis, OR (US)

(73) Assignee: Oregon State University, Corvallis, OR (US)

(21) Appl. No.: 18/334,237

(22) Filed: Jun. 13, 2023

Related U.S. Application Data
(60) Provisional application No. 63/366,450, filed on Jun. 15, 2022.

(51) Int. Cl. G01N 27/327; G01N 27/416

(52) U.S. Cl. CPC G01N 27/3277; G01N 27/4161

(57) ABSTRACT

Disclosed herein is an electrochemical biosensor comprising at least one working electrode on an electrically insulative substrate. At least one working electrode incorporates an electrocatalytic film, comprising a molecularly imprinted polymer (MIP) layer. In at least one embodiment, MIP layer comprises a plurality of shape-selective cavities and a plurality of non-biological electrocatalytic centers.

### CLAIM OF PRIORITY

This application claims priority to U.S. Provisional Patent Application No. 63/366,450, filed Jun. 15, 2022, titled “Electrocatalytic Polymer Devices For Biological Detection,” which is incorporated by reference in its entirety.

### STATEMENT OF GOVERNMENT INTEREST

This invention was made with government support under grant ECCS1810067 awarded by the National Science Foundation. The government has certain rights in the invention.

### BACKGROUND

In recent years, electrochemical biosensors have enjoyed good success at detection and quantification of diverse biological samples, including macromolecules, whole viruses and cells. Electrochemical biosensors are amenable to microfabrication methods and integration with monolithic MEMS devices, thus can be manufactured en masse inexpensively. Many examples include thin-film electrochemical cells comprising metallic electrodes coated on plastic substrates by well-known lithographic methods. Electrochemical biosensors have proven to be highly sensitive, capable of detecting femtomolar, picomolar and nanomolar concentrations of analytes in many cases. Electrochemical detection is generally based on active redox centers within these biological materials. The active redox centers may directly or indirectly interact with a working electrode. Without a specific binding mechanism, the surface of the working electrode of electrochemical sensors can be inherently non-specific. Many substances present in solution, including water, may react (e.g., undergo oxidation or reduction) with a working electrode at a given potential. To overcome the inherent lack of specificity, organic layers, ranging from self-assembled monolayers to polymer films, have been coated or adsorbed on electrode surfaces. Such organic layers may provide a scaffold for specific binding sites that can bind solution-phase target analytes to an electrode surface with a high degree of specificity. Examples include biologically active antibodies and enzymes tethered on and/or within the organic layer. These immobilized biological binding sites functionalize the organic layer and enable target analytes to adsorb with high specificity on the electrode surface. Redox chemistry may occur directly at the electrode, or indirectly by an intermediary redox molecule that first reacts at the electrode surface and then with the analyte.

In many cases, the biological binding sites may exhibit thermal instability and are subject to fouling. In addition, the enzymes and antibodies are generally costly and difficult to handle. Recently, molecularly imprinted polymers (MIPs) coated on working electrode surfaces have found a great deal of success enhancing specificity of electrochemical biosensors without the need to immobilize biologically active molecules. MIPs make use of template molecules to form shape-specific cavities within a polymer matrix during synthesis. The template molecules may be themselves those of one or more intended target analytes. Thus, MIP layers may be readily tailored for detection of one or more specific target analytes. During operation of a MIP-bearing electrochemical biosensor, a target analyte in solution may encounter the MIP layer and diffuse into the polymer matrix. Molecules of the target analyte may encounter and diffuse into the shape-specific cavities tailored to fit the target molecule, thereby binding with high affinity to the MIP layer.

At present, electrochemical biosensors employing MIP layers utilize solution phase or tethered redox probes. The redox probes may be of biological origin and are inherently unstable, short lived and relatively expensive. Even non-biological redox probe technologies often require complicated work-ups and may not be generally amenable to point of care sensors in medical applications.

### BRIEF DESCRIPTION OF THE DRAWINGS

FIG. 1A illustrates a single layer electrocatalytic film comprising a single MIP layer, in accordance with at least one embodiment.

FIG. 1B illustrates a single layer electrocatalytic film comprising single MIP layer covered by an insulative layer as a second layer over the MIP layer, in accordance with at least one embodiment.

FIG. 1C illustrates a multilayer electrocatalytic fil, in accordance with at least one embodiment.

FIG. 1D illustrates an electrocatalytic film in accordance with at least one embodiment.

FIG. 2A illustrates an upper plan view of an electrochemical biosensor, in accordance with at least one embodiment.

FIG. 2B illustrates a lower plan view of the electrochemical biosensor shown in FIG. 2A, in accordance with at least one embodiment.

FIGS. 3A-3D illustrate a target analyte detection scheme using an electrochemical biosensor, in accordance with at least one embodiment.

FIG. 4 illustrates a family of cyclic voltammograms comparing cyclic voltammograms of an EC-P-coated working electrode, in accordance with at least one embodiment.

FIG. 5 illustrates a family of chronoamperometric curves comparing chronoamperometric responses of a working electrode in the presence of cortisone and cortisol as target analytes, in accordance with at least one embodiment.

FIG. 6A illustrates a progression of chronoamperometry response curves for a working electrode comprising cortisol binding sites, in accordance with at least one embodiment.

FIG. 6B illustrates a progression of chronoamperometry response curves for a working electrode comprising polymer without binding sites, in accordance with at least one embodiment.

FIG. 7 illustrates a binding isotherm for cortisol and cortisone on molecular imprinted polymer (MIP)-coated working electrodes and non-MIP coated working electrodes, in accordance with at least one embodiment.

FIG. 8 illustrates binding isotherms of cortisol and other analytes on an MIP film-coated working electrode, in accordance with at least one embodiment.

FIG. 9 illustrates an exemplary process flow of a method of producing the disclosed electrochemical biosensor, in accordance with at least one embodiment.

FIG. 10A illustrates a process flowchart summarizing an exemplary method for using an electrochemical biosensor in a biological assay, in accordance with at least one embodiment.

FIG. 10B illustrates an exemplary purge/renewal process by dissociation of the bound target analyte molecules, in accordance with at least one embodiment.

FIG. 11 illustrates a molecular structure of copper phthalocyanine tetrasulfonate (CuPcTS), in accordance with at least one embodiment.

FIG. 12 illustrates a block diagram of a system comprising a reader for an electrochemical biosensor in accordance with at least one embodiment.

### DETAILED DESCRIPTION

Disclosed herein is an integrated electrochemical biosensor comprising a catalytic film over one or more working electrodes, in accordance with at least one embodiment. In at least one embodiment, catalytic film comprises electrocatalyst molecules embedded in a molecularly imprinted polymer (MIP) matrix. In at least one embodiment, MIP matrix comprises shape-selective cavities for high-affinity selective binding of target analytes. In at least one embodiment, the electrocatalyst molecules may be in close proximity of analyte molecules trapped within the shape-selective cavities within the MIP matrix. In at least one embodiment, the catalytic film comprises one or more layers. In at least one embodiment, one layer of the one or more layers comprises a molecularly imprinted polymer (MIP). In at least one embodiment, one layer of the one or more layers comprises an immobilized electrocatalyst. In at least one embodiment, the electrocatalytic film comprises a single chemically homogeneous layer. In at least one embodiment, single homogeneous layer comprises an MIP, where shape-specific cavities within a polymer matrix have been formed by template molecules of the target analyte. In at least one embodiment, single homogeneous layer further comprises an immobilized electrocatalyst embedded within the same polymer matrix.

The disclosed electrochemical biosensor may be employed as a point of care device for rapid detection of target analytes, in accordance with at least one embodiment. In at least one embodiment, analytes may include, but are not limited to, steroid hormones, drugs or metabolites thereof and other biomarkers present in body fluids such as sweat, saliva, blood plasma and urine. In at least one embodiment, target analytes may be glucocorticoid hormones cortisone and cortisol. In at least one embodiment, target analytes are stress hormones related to physical and psychological stress. In at least one embodiment, overabundance or deficiency of cortisol, for example, may be a biomarker for a variety of abnormalities, such as Addison's disease, Cushing's disease, post-traumatic stress disorders (PTSD) and chronic fatigue syndrome.

In at least one embodiment, an electrocatalytic film comprises multiple layers in a chemically stratified structure. In at least one embodiment, a first layer comprises immobilized electrocatalyst molecules physisorbed or chemically tethered to an electrochemically active surface of a working electrode. In at least one embodiment, a second layer comprises an MIP. In at least one embodiment, MIP may be as described for the homogenous catalytic film.

In at least one embodiment, a second or third layer is over the MIP layer in both single and multiple layer embodiments, respectively. In at least one embodiment, the second or third layer may comprise a pure polymer matrix having no shape-selective cavities nor bound catalyst. In at least one embodiment, the second or third layer may comprise a MIP having shape-selective cavities, but no bound catalyst. In at least one embodiment, third layer may be inactive chemically, providing an insulating layer to mitigate any electrochemical response from non-specifically adsorbed analytes.

In at least one embodiment, the insulating layer (second or third layer over the layer containing the electrocatalyst) may comprise the same polymer as the MIP layer (e.g., PPy). In at least one embodiment, the insulating layer may comprise a heteromaterial (e.g., different from the MIP layer). In at least one embodiment, insulating layer may comprise any of polyethylene glycol, polyglycerol, cellulose, agarose, hyaluronic acid, Nafion, zwitterionic polymers comprising sulphobetaine, carboybetaine, phophonobetaine, or phosphorylcholine.

In at least one embodiment, a third layer may help to insulate the electrode from non-specifically bound molecules on or in the MIP layer that may cause false positive signals. In at least one embodiment, non-specifically bound analytes may be closely related to the target analyte, including the target analyte itself, or chemically non-analogous molecules.

In at least one embodiment, the disclosed electrochemical biosensor obviates employment of expensive and short-lived biological redox enzymes, antibodies and aptamers by employment of non-biological metal-based electrocatalytic centers in the form of macrocyclic complexes of suitable metal redox centers. Biological molecules such as enzymes have limited stability and are relatively expensive to acquire. Macrocyclic metal complex electrocatalytic centers, such as copper phthalocyanine, can substitute for redox enzymes. Such non-biological electrocatalytic centers can provide a redox activity toward target analytes that is similar to some redox enzymes, such as the activity hydroxysteroid dehydrogenase exhibits towards corticosteroids.

In at least one embodiment, the disclosed electrochemical biosensor affords high selectivity toward target analyte molecules by incorporation of molecular imprinted polymer (MIP) layers on one or more integrated working electrodes. The MIP layer comprises molecularly imprinted shape-selective cavities that provide high-affinity binding sites for target analytes that fit within the shape-selective cavities. The MIP layers may be produced by inclusion of target analyte molecules in a layer precursor mixture and form the cavities during polymerization of the precursor mixture. The combination of high specificity afforded by incorporation of a MIP layer, and high redox activity of the non-biological electrocatalytic centers enables the disclosed electrochemical biosensor to have substantially equivalent performance of conventional electrochemical biosensors incorporating biological molecules. However, the relatively low cost and greater longevity of the disclosed electrochemical biosensor may be significantly advantageous in comparison to biologically based electrochemical biosensors.

In at least one embodiment, the disclosed electrochemical biosensor enables direct redox of a target analyte without the use of intermediary redox probes. For example, the electrocatalytic centers may directly reduce ketone or aldehyde groups, or oxidize hydroxyl groups. In contrast, some conventional electrochemical biosensors may employ redox intermediates such as ferrocyanide in solution for indirect determination of target analytes. Some conventional electrochemical biosensors also incorporate MIP layers, in which target analytes bind. The electrochemistry in these devices is based on reduction or oxidation of the redox probe. When target analyte binds to the MIP layer, the electrochemical current is reduced due to impeded access and hinderance of the electron transfer pathway of the redox probe to or from the working electrode. Thus, the higher the concentration of target analyte, the lower the electrochemical current. Such biosensors are subject to false readings, interference and cross-reactivity.

In the following description, well-known methods and devices are shown in block diagram form, rather than in detail, to avoid obscuring the present disclosure. Reference throughout this specification to “an embodiment” or “one embodiment” “at least one embodiment,” or “some embodiments” means that a particular feature, structure, function, or characteristic described in connection with the embodiment is included in at least one embodiment of the disclosure. The appearances of the phrase “in an embodiment,” “in at least one embodiment,” “in one embodiment,” or “some embodiments” in various places throughout this specification are not necessarily referring to the same embodiment of the disclosure. Furthermore, the particular features, structures, functions, or characteristics may be combined in any suitable manner in one or more embodiments. For example, a first embodiment may be combined with a second embodiment anywhere the particular features, structures, functions, or characteristics associated with the two embodiments are not mutually exclusive.

Here, “electrochemical biosensor” may generally refer to an integrated sensor comprising an electrochemical cell comprising one or more working electrodes, a reference electrode and a counter electrode. The electrochemical cell may be operable to have a high selectivity to a target analyte, which may be a biologically active compound, such as a steroid, other hormone, metabolite, etc. that is amenable to undergo an electrolytic reaction driven by the electrochemical cell.

Here, “working electrode” may generally refer to an electrode of an electrochemical cell that is poised at a potential to carry out an electrolytic reaction. An electrolytic reaction may be an electrochemical reaction that is driven by a potentially applied to the working electrode. The electrolytic reaction may be reversible or irreversible. The potential of a working electrode may be controlled by a potentiostat and may be measured relative to a known redox couple, which may form a reference electrode. For example, a reference electrode may employ a silver/silver chloride couple. A working electrode may be a cathode or an anode in an electrochemical cell depending on the potential of the electrode and the reaction undergoing reduction or oxidation, respectively.

Here, “electrochemically active surface” may generally refer to a surface of a working electrode upon which electrochemical reactions take place. The electrochemically active surface may be a surface of the working electrode that is exposed to an electrolyte.

Here, “reference electrode” may generally refer to an electrode of an electrochemical cell that is poised at a potential set by a known redox couple. An example is a silver/silver chloride (Ag/AgCl) electrode. The Ag/AgCl electrode may comprise a solid silver wire or plate that is coated by a layer of solid AgCl. A steady potential is set up by the couple, (eg., The solid phase of the Ag/AgCl electrode enables it to be particularly convenient for applications in integrated electrochemical sensors.

Here, “counter electrode” may generally refer to an electrode of an electrochemical cell that serves as a cathode or anode in opposition to the working electrode. The counter electrode may complete the electrochemical “circuit” in the electrochemical cell.

Here, “electrically insulative substrate” may generally refer to a sheet substrate for an integrated biosensor that is made from an electrically insulative material, such as a plastic, glass or ceramic material. The insulative substrate may provide mechanical support for the electrochemical biosensor according to at least one embodiment. For example, the electrochemical cell may be formed on an insulative substrate by depositing a patterned thin film comprising a conductive material on the insulative substrate.

Here, “molecular imprinted polymer (MIP) layer” may generally refer to a polymer layer that comprises shape-selective cavities that are formed by an imprint process. In at least one embodiment, shape-selective cavities may be formed by molecular template molecules trapped within a polymer layer during polymerization, and subsequently eluted or otherwise liberated to leave cavities behind in the film. The cavities can have similar dimensions and shape as the template molecules. The MIP layer has a high binding affinity for the template molecules. Chemical analogs of the template molecules may have a lesser binding affinity. Thus, a MIP can be highly selective for template molecule binding.

Here, “shape-selective cavities” may generally refer to cavities created in a MIP film when embedded template molecules are liberated. As noted, shape-selective cavities impart high binding affinity for adsorption of template molecules on and within a MIP film.

Here, “electrocatalytic center” may generally refer to a redox catalyst comprising a metal atom center, generally comprising a transition metal, where the metal atom is chelated or complexed to a macrocyclic ring. An electrocatalytic center may mimic an active center of an enzyme.

Here, “non-biological catalyst” may generally refer to a non-biological molecule that may have a catalytic function that works in a similar manner as a biological enzyme.

Here, “phthalocyanine ring” may generally refer to a macrocyclic ring having coordination moieties such as amino groups that chelate a metal atom center. The phthalocyanine ring comprises four isoindole rings that form N-coordination bonds to the metal atom center.

Here, “ionizable sidechain” may generally refer to ionizable groups that are attached to a phthalocyanine ring. Ionizable sidechains may comprise oxo, amino, carboxylic acid, sulfate, sulfonate, phosphate, phosphonate, groups that may acquire a charge by adjustment of the local pH.

Here, “metal atom” may generally refer to a transition metal or a Group IIA metal complexed with a macrocyclic ring. The metal atom may function as a redox center within the macrocyclic complex such as a phthalocyanine ring.

Here, “single-atom catalyst” may generally refer to a catalytic molecule that has a single atom that functions as the active center of the catalytic molecule.

Here, “metal-organic framework (MOF)” may generally refer to a catalyst that comprises an extended molecular structure, such as a graphite or graphene network, carbon nanotubes, etc., that may intercalate metal atoms that perform as active centers, such as redox centers.

Here, “non-selective, non-catalytic” may generally refer to a polymer layer that does not have catalytic properties nor a large binding affinity for any particular molecular species. For example, a non-MIP polymer comprising polypyrrole without shape-selective cavities nor electrocatalytic centers is non-catalytic nor non-selective in that it does not have a high binding affinity for a particular molecular species and does not catalyze chemical or electrochemical reactions.

Here, “interconnect pads” may generally refer to integrated electrical contacts patterned on an edge of an electrochemical biosensor substrate. The interconnect pads may enable insertion of the electrochemical biosensor into a connector interface to a device such as an electrochemical biosensor reader, and interconnection of electrodes on the biosensor to a potentiostat and heater controller in the reader.

Here, “interface” may generally refer to a connector structure that may receive an inserted electrochemical biosensor. Interconnect pads on the edge of the biosensor may interface with contacts in the interface.

Here, “potentiostat circuitry” may generally refer to circuitry within a reader that is operable to perform as a potentiostat. In at least one embodiment, the potentiostat circuitry is interfaced to electrodes on the electrochemical biosensor.

Here, “temperature controller circuitry” may generally refer to circuitry with a reader that is operable to perform as a temperature controller. In at least one embodiment, the temperature controller circuitry is interfaced to a microheater on the electrochemical sensor.

Here, “target analyte” may generally refer to a substance that is of interest to be measured by an electrochemical sensor. A target analyte may be cortisol in a bodily fluid, for example. The target analyte may have a high binding affinity with a MIP layer as the target analyte is also the template substance employed in the formation of the shape-selective cavities with in the MIP layer.

Here, “concentration of the target analyte” may generally refer to the concentration of the target analyte within the bodily fluid.

Here, “electrochemical potential” may generally refer to a voltage imposed on a working electrode to excite an electrochemical reaction on the working electrode surface.

Here, “chronoamperometric method” may generally refer to an electrochemical measurement method where an electrode is poised at a steady potential by stepping the electrode potential from one voltage to another voltage and measuring the electrochemical current after a it reaches a quasi-steady state after an initial decay period. The current may be recorded after a period of several seconds, for example. The current may be related to the concentration of target analyte in solution, for example.

Here, “electrochemical potential” may generally refer to a voltage imposed on a working electrode by a potentiostat and measured against a reference electrode. The electrochemical potential may be any voltage at which the working electrode is held vs. the reference electrode. Electrochemical potentials are states in volts vs. a reference electrode, such as a Ag/AgCl electrode.

Here, “electrochemical current” may generally refer to an electrical current that flows to or from a working electrode when the working electrode is poised at a potential by a potentiostat. The electrochemical current is measurable by the potentiostat.

Here, “differential current” may generally refer to a difference between two electrochemical currents. For example, a first electrochemical current may be measured at a first working electrode that has an MIP layer for binding a target analyte. A second electrochemical current may be measured at a second working electrode that has a non-specific polymer coating. A differential current may be determined by subtracting the second current from the first current to remove the effect of non-specific adsorption on the electrochemical current measured by the first working electrode.

Here, “non-specific polymer” may generally refer to a polymer that has no binding sites, such as shape-specific cavities, for any particular molecule.

Here, “microheater” may refer to an integrated heating element patterned on an electrochemical biosensor substrate in accordance with at least one embodiment. The microheater may heat working electrodes on the biosensor to elevated temperatures to purge the MIP layers on the working electrodes of bound analyte, for example.

Here, terms “coupled” and “connected” along with their derivatives, may be used to describe functional or structural relationships between components. These terms are not intended as synonyms for each other. Rather, in at least one embodiment, “connected” may be used to indicate that two or more elements are in direct physical, optical, or electrical contact with each other. Here, “coupled” may be used to indicate that two or more elements are in either direct or indirect (with other intervening elements between them) physical, electrical or in magnetic contact with each other, and/or that the two or more elements co-operate or interact with each other (e.g., as in a cause an effect relationship). Here, “coupled” may also generally refer to direct attachment of one electronic component to another. An electric or magnetic field may couple one component to another, where the field is controlled by one component to influence the other in some manner.

Here, “over,” “under,” “between,” and “on” may generally refer to a relative position of one component or material with respect to other components or materials where such physical relationships are noteworthy. Unless these terms are modified with “direct” or “directly,” one or more intervening components or materials may be present. Similar distinctions are to be made in the context of component assemblies. As used throughout this description, and in the claims, a list of items joined by “at least one of” or “one or more of” can mean any combination of the listed terms.

Here, “substantially,” “close,” “approximately,” “near,” and “about,” generally refer to being within +/−10% of a target value. For example, unless otherwise specified in explicit context of their use, terms “substantially equal,” “about equal” and “approximately equal” mean that there is no more than incidental variation between among things so described. In at least one embodiment, such variation is no more than +/−10% of a predetermined target value.

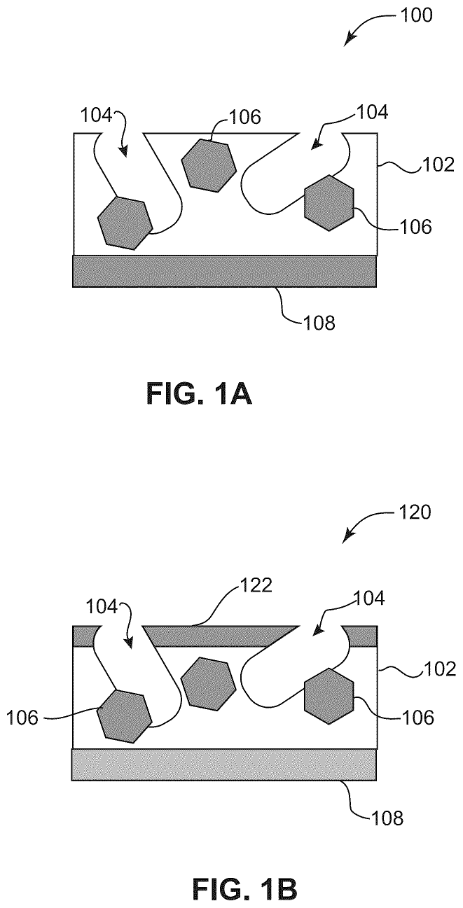

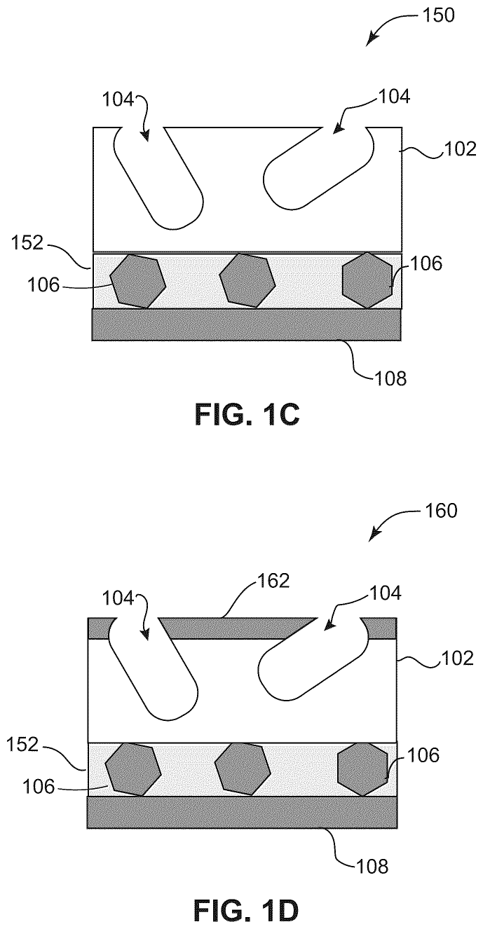

FIG. 1A illustrates a single layer electrocatalytic film 100 comprising single MIP layer 102, in accordance with at least one embodiment. In at least one embodiment, MIP layer 102 comprises a plurality of imprinted shape-selective cavities 104, shown dispersed within the polymer matrix of MIP layer 102. In at least one embodiment, MIP layer 102 comprises a suitable polymer, such as polypyrrole (PPy) or poly dimethylpyrrole. In at least one embodiment, a polypyrrole film may be produced by polymerization of a pyrrole monomer mixed with one or more molecular templates (e.g., cortisol). In at least one embodiment, MIP layer 102 can be synthesized by thermal, photopolymerization or electropolymerization techniques. In at least one embodiment, MIP layer synthesis may be a solution-phase polymerization or a heterogeneous-phase polymerization. In at least one embodiment, a thermal, photo- or electropolymerization of the MIP layer may be performed directly on a working electrode (e.g., electrode 108, described below). In at least one embodiment, MIP films may be synthesized by polymerization of suitable monomers (e.g., pyrrole) in the presence of molecules of a target analyte template substance (e.g., cortisol) to imprint shape-selective cavities within the polymer matrix of the MIP. In at least one embodiment, polymers comprising pure monomers or mixtures of monomers may be suitable for specific implementations.

In at least one embodiment, MIP layer 102 may be synthesized from monomers such as, but not limited to, pyrrole, dimethylpyrrole, phenol, o-aminophenol, o-phenylenediamine, aniline, scopoletin, 2,4-ethylenedioxythiophene, dopamine, monomers with boronic acid groups, vinyl monomers, and acrylates. In at least one embodiment, after polymerization, template molecules are removed to leave shape-selective cavities 104, completing MIP layer 102. In at least one embodiment, MIP layer 102 has a thickness that may range between 5 nm to 1 micron. In at least one embodiment, MIP layer 102 has a thickness of less than 200 nm.

In at least one embodiment, MIP layer 102 comprises a plurality of electrocatalytic centers 106, here represented by shaded hexagons distributed within MIP layer 102. In at least one embodiment, electrocatalytic centers 106 can comprise suitable non-biological redox centers enabling reversible redox chemistry. In at least one embodiment, reversible redox centers may include, but not be limited to, transition metals such as magnesium (Mg), aluminum (Al), zinc (Zn), copper (Cu), iron (Fe), cobalt (Co), manganese (Mn), nickel (Ni), and ruthenium (Ru). In at least one embodiment, transition metal redox centers may be complexed by primary and/or secondary amino groups within a macrocyclic metal complex type structure, such as a metalloporphyrin or metallophthalocyanine ring.

In at least one embodiment, the macrocyclic ring core may have attached sidechains. Sidechains may be neutral or charged. In at least one embodiment, a sidechain may have an ionizable moiety such as sulphonate group, a phosphate or phosphonate group, a carboxylic acid or an amino group. In at least one embodiment, electrocatalyst may comprise a phthalocyanine ring core having ionizable sulphonic acid groups attached to at least one of four isoindole units of the phthalocyanine core. In at least one embodiment, charged sidechains may comprise ionizable groups such as sulphonate groups, phosphonate groups, amino groups or carboxylic acid groups. In at least one embodiment, sulphonic acid groups may be ionized to form sulphonate anions by regulation of the pH of an aqueous solution, for example. In at least one embodiment, incorporation of charged sidechains may enhance the electrical conductivity of the host polymer of the catalytic film. In at least one embodiment, if the host polymer comprises polypyrrole (PPy), the native electrical conductivity of PPy may be limited to residual low electronic conductivity of the extended pi-bond system within the polymer. In at least one embodiment, charged sidechains may contribute to electrical conductivity by providing cations or anions formed by ionizable groups attached to the phthalocyanine core.

In at least one embodiment, phthalocyanine ring may afford higher complexation stability with metal atoms such as copper than other macrocyclic ring systems such as porphyrin rings found in naturally occurring heme and chlorophyll. In at least one embodiment, due to more extensive pi electron conjugation, redox efficiency of the metal atom center is greater in phthalocyanine rings than in naturally occurring porphyrin systems. In at least one embodiment, CuPcTS as a non-biological electrocatalytic center may mimic kinetics and selectivity of some redox enzymes, for example, hydroxysteroid dehydrogenase. This enzyme catalyzes the interconversion of cortisol and cortisone in humans. In at least one embodiment, CuPcTS is employed to catalyze the reduction (hydrogenation) of cortisol to hydrogenated cortisol, where the keto groups at carbon 3 and carbon 20 of cortisol are reduced to the corresponding hydroxyl groups at these same positions.

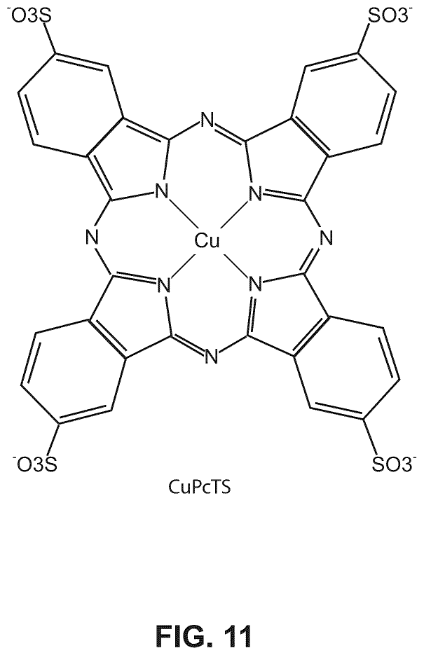

In at least one embodiment, ionizable groups may be acids or bases such as sulphonates, phosphonates, boronic acids, carboxylic acids, and amino groups. In at least one embodiment, charged sidechains may enhance incorporation of electrocatalysts into a polymer matrix during polymerization of the MIP. In at least one embodiment, incorporation of neutral electrocatalysts may require additional reagents, such as surfactants, for incorporation into the polymer matrix during polymerization. In at least one embodiment, charged sidechains may enhance conductivity of the MIP polymer matrix during operation of the biosensor. In at least one embodiment, degree of ionization may depend in part on the pKa of the ionizable group attached to the phthalocyanine core. In at least one embodiment, the phthalocyanine core comprises a copper atom. An exemplary metallophthalocyanine molecule is copper phthalocyanine tetrasulphonate (CuPcTS). A structure of CuPcTS is shown in FIG. 11, in accordance with at least one embodiment. In at least one embodiment, CuPcTS comprises four ionizable sulphonate groups on the CuPc core. In at least one embodiment, while ionizable sulphonate groups may afford some advantages in particular applications, other ionizable groups such as phosphonates, carboxylic acids and amino groups may also be suitable candidates for charged sidechains on the phthalocyanine core.

In at least one embodiment, non-biological metallophthalocyanines such as CuPcTS may mimic the selectivity and electro catalytic activity of some enzymes, such as hydroxysteroid dehydrogenase, for example. In at least one embodiment, contrasted non-biological electrocatalysts, metallophthalocyanines such as CuPcTS are thermally stable and relatively inexpensive. In at least one embodiment, these electrocatalytic compounds are readily immobilized within an MIP by chemical bonds or by van der Waals forces. For the latter, CuPcTS may be embedded within the polymer matrix of the MIP. Details of synthesis of the MIP and incorporation of CuPcTS within the polymer matrix are described below.

In at least one embodiment, the macrocyclic ring may comprise sulfone, amino, carboxylic acid or phosphate side groups. In at least one embodiment, electrocatalytic centers 106 comprise copper phthalocyanine tetrasulfonate or analogs thereof. In at least one embodiment, electrocatalytic centers 106 are dispersed within the polymer matrix of MIP layer 102 or on a surface of electrode 108 supporting MIP layer 102. In at least one embodiment, electrocatalytic centers 106 may be electrodeposited, entrapped, or covalently bonded to the polymer matrix of MIP layer 102, and/or covalently bonded or physisorped on electrode 108.

In at least one embodiment, electrocatalytic centers 106 comprise single atom catalysts, comprising a metal atom embedded on a nitrogen-doped carbon matrix or on a carbon matrix containing metal-nitrogen-carbon sites. In at least one embodiment, electrocatalytic centers 106 comprise metal-organic frameworks MOFs (e.g., a metal-terephthalate MOF). In at least one embodiment, electrocatalytic centers 106 can comprise transition metal elements such as manganese (Mn), iron (Fe), cobalt (Co), nickel (Ni), copper (Cu), zine (Zn), magnesium (Mg), aluminum (Al) and ruthenium (Ru), noble metals such as gold (Au) silver (Ag), platinum (Pt), palladium (Pd), metal oxides, basal-plane pyrolytic graphite, graphene, β-cyclodextrin, and two-dimensional nanomaterials. In at least one embodiment, electrocatalytic centers 106 may be entrapped, electrodeposited, or chemically tethered within MIP layer 102 or on the surface of the working electrode (e.g., electrode 108, described below).

In at least one embodiment, MIP layer 102 is attached to the surface of electrode 108. In at least one embodiment, MIP layer 102 is covalently bonded to electrode 108. In at least one embodiment, MIP layer 102 may be physisorbed onto electrode 108. In at least one embodiment, electrode 108 comprises a suitable metal or conductive material, such as platinum, palladium, silver, gold, indium tin oxide, and carbon (e.g., glassy carbon, carbon nanotube, carbon dot), graphite, graphene, or granulated carbon particles suspended within a matrix). In at least one embodiment, electrode 108 comprises a metal layer, such as gold, platinum or carbon coated on a non-conducive substrate. In at least one embodiment, electrode 108 can be formed on an organic polymer substrate, such as polyethylene terephthalate (PET) or polycarbonate, or an inorganic material such as silicate glass, aluminum oxide or aluminum nitride. The substrate may provide mechanical support to electrode 108, and not participate in electrochemical processes.

In at least one embodiment, target molecules employed to form shape-selective cavities 104 may be compounds that can undergo reversible and irreversible redox chemistries. In at least one embodiment, target molecules may include hydroxyl, thiol and/or phenol groups. In at least one embodiment, exemplary compounds may include, but not be limited to, steroid hormones such as cortisol, progesterone, estradiol, and/or drugs, such as tetrahydrocannabinol, morphine, cocaine, codeine, methamphetamine, as well as metabolites of any of the afore-mentioned compounds. In at least one embodiment, exemplary target molecules may further include neurotransmitters such as dopamine, serotonin, norepinephrine and epinephrine. In at least one embodiment, exemplary target molecules may further include carbohydrates such as glucose and fructose. In at least one embodiment, target molecules may further include antibiotics (e.g., tetracycline), alcohols, cholesterol, vitamins (e.g., ascorbic acid, vitamin B1), amino acids, antioxidants (e.g., N-acetylcysteine, glutathione) and inorganic compounds such as hydrazine and hydroxylamine. In at least one embodiment, exemplary target compounds may further include peptides, proteins, nucleotides, whole or partial eucaryotic cells, viruses, bacteria, pesticides, herbicides, fungicides, insecticides, toxins, and pollutants.

FIG. 1B illustrates single layer electrocatalytic film 120 comprising single MIP layer 102 covered by insulative layer 122 as a second layer is over MIP layer 102, which is on the electrode surface. In at least one embodiment, electrocatalytic film 120 is a two-layer stratified structure. In at least one embodiment, electrocatalytic centers 106 are confined to MIP layer 102 under insulative layer 122. In at least one embodiment, insulative layer 122 comprises a native material from MIP layer 102, such as a polypyrrole, or other materials such as polyethylene glycol, polyglycerol, cellulose (e.g., agarose or hyaluronic acid), Nafion, zwitterionic polymers containing sulfobetaine, carboxybetaine, phosphonobetaine or phosphorylcholine. In at least one embodiment, insulative layer 122 may comprise a polymer matrix without inclusion of shape selective cavities or electrocatalytic centers. In at least one embodiment, insulative layer 122 may be synthesized by polymerization of a pure monomer or monomer mixture in the absence of target molecules. In at least one embodiment, insulative layer 122 may prevent non-specific adsorption of non-target substances on MIP layer 102. Such non-target substances may interfere with target molecules, resulting in inaccurate measurements. In at least one embodiment, shape-selective cavities 104 may enable selective adsorption of target molecules by capture of target molecules having shape and dimensions substantially matching those of shape-selective cavities 104.

FIG. 1C illustrates multilayer electrocatalytic film 150, in accordance with at least one embodiment. In at least one embodiment, electrocatalytic film 150 is an example of a multilayer catalytic film having a stratified structure. In at least one embodiment, electrocatalytic film 150 comprises MIP layer 102 over electrocatalyst layer 152. In at least one embodiment, electrocatalyst layer 152 is immediately adjacent to electrode 108. In at least one embodiment, MIP layer comprises shape-selective cavities 104 dispersed within MIP layer 102. In at least one embodiment, electrocatalytic centers 106 are substantially confined within electrocatalyst layer 152 and are separated from MIP layer 102. As shown in FIG. 1C, electrocatalyst layer 152 is confined to a first layer closest to the electrode surface. MIP layer 102 comprises a second layer over the first layer comprising electrocatalyst layer 152. In at least one embodiment, electrocatalyst layer 152 comprises substantially the same polymer as MIP layer 102. In at least one embodiment, electrocatalytic centers 106 may be physisorbed or chemisorbed onto the electrode surface. In at least one embodiment, electrocatalytic centers 106 are electrodeposited on electrode 108.

FIG. 1D illustrates electrocatalytic film 160, in accordance with at least one embodiment. In at least one embodiment, electrocatalytic film 160 is a multilayer film structure comprising insulative layer 162, shown as a third layer over MIP layer 102. In at least one embodiment, insulative layer 162 may have a similar function as insulative layer 122 described for FIG. 1B.

In at least one embodiment, electrocatalytic centers 106 are confined electrocatalytic layer 152. In at least one embodiment, insulative layer 162 comprises a native material from MIP layer 102, such as a polypyrrole, or other materials such as polyethylene glycol, polyglycerol, cellulose (e.g., agarose or hyaluronic acid), Nafion, zwitterionic polymers containing sulfobetaine, carboxybetaine, phosphonobetaine or phosphorylcholine. In at least one embodiment, insulative layer 162 may comprise a polymer matrix without inclusion of shape selective cavities or electrocatalytic centers. In at least one embodiment, insulative layer 162 may be synthesized by polymerization of a pure monomer or monomer mixture in the absence of target molecules. In at least one embodiment, insulative layer 162 may prevent non-specific adsorption of non-target substances on MIP layer 102. Such non-target substances may interfere with target molecules, resulting in inaccurate measurements.

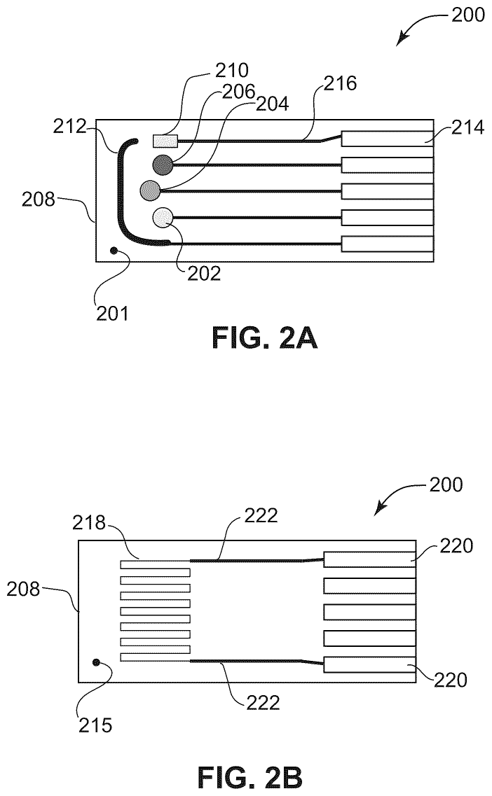

FIG. 2A illustrates an upper plan view of electrochemical biosensor 200, in accordance with at least one embodiment. In at least one embodiment, upper surface 201 of electrochemical biosensor 200 comprises one or more working electrodes (e.g., working electrodes 202, 204 and 206) patterned on substrate 208. While three working electrodes are shown, in at least one embodiment, any suitable number of working electrodes may be employed. In at least one embodiment, electrochemical biosensor 200 may comprise one working electrode or five working electrodes. In at least one embodiment, multiple working electrodes may enable several analytes (target substances) to be detected by a single electrochemical biosensor 200. In at least one embodiment, electrochemical sensor further comprises reference electrode 210 patterned on substrate 208. In at least one embodiment, working electrodes 202, 204, and 206 comprise a conductive base patterned on substrate 208. In at least one embodiment, the conductive base may comprise a noble metal such as gold, silver, platinum, palladium. In at least one embodiment, the conductive base may comprise indium tin oxide. In at least one embodiment, the conductive base may comprise glassy carbon, or a conductive suspension comprising carbon nanotubes, graphene, or carbon dots. In at least one embodiment, working electrodes 202, 204, and 206 comprise a molecular imprinted polymer (MIP) layer to enable analyte specificity. In at least one embodiment, an MIP layer may be coated over a conductive base, as described below.

In at least one embodiment, reference electrode 210 comprises a conductive base coated with a solid electrochemical couple. In at least one embodiment, electrochemical couple is a silver (Ag) metal electrode base coated by a layer of silver chloride (AgCl). In at least one embodiment, reference electrode 210 is positioned to be adjacent to any of working electrodes 202, 204, or 206. In at least one embodiment, electrochemical biosensor 200 further comprises counter electrode 212. In at least one embodiment, counter electrode 212 comprises a noble metal such as gold, silver, platinum, or palladium. In at least one embodiment, counter electrode 212 may comprise glassy carbon, or a conductive suspension comprising carbon nanotubes, graphene, or carbon dots. In at least one embodiment, counter electrode 212 comprises indium tin oxide.

In at least one embodiment, working electrodes 202-206, reference electrode 210 and counter electrode 212 may be formed on substrate 208 by screen printing techniques or by thin film deposition processes such as vacuum sputtering or evaporation. In at least one embodiment, conductive layers of working electrodes 202-206, reference electrode 210 and counter electrode 212 may have thicknesses ranging from 100 nm to several microns.

In at least one embodiment, counter electrode 212 may have a geometry and position engineered for optimal current distribution between any of working electrodes 202-206 and counter electrode 212. In at least one embodiment, counter electrode 212 has a wide arc proximal to working electrodes 202-206. In at least one embodiment, counter electrode 212 may have a substantially greater surface area than working electrodes 202-206 to mitigate high current densities and overpotentials due to mass transfer limitations.

In at least one embodiment, working electrodes 202-206, reference electrode 210 and counter electrode 212 are coupled to interconnect pads 214 by traces 216, where interconnect pads 214 and traces 216 are integrated (e.g., patterned) on substrate 208. In at least one embodiment, interconnect pads 214 and traces 216 may comprise the same conductive material as used for working electrodes 202-206. In at least one embodiment, interconnect pads 214 and traces 216 may comprise a conductive material such as copper or aluminum. In at least one embodiment, interconnect pads 214 may enable electrochemical biosensor 200 to be inserted into a connector socket having corresponding receptacle contacts. In at least one embodiment, such a connector socket may be part of a point-of-care or similar device to activate and read electrochemical biosensor 200.

In at least one embodiment, electrochemical detection of target analyte molecules selectively bound to working electrodes 202-206 may be performed by electrochemical biosensor 200. In at least one embodiment, target analyte detection may be performed by measurement of electrochemical currents (e.g., by chronoamperometry) corresponding to reduction or oxidation or target analytes at fixed electrode potentials (e.g., with respect to the reference electrode potential). In at least one embodiment, magnitudes of response currents may be correlated with concentration levels of target analytes within bodily fluid samples. In at least one embodiment, a sample of body fluid, such as blood plasma, urine, breath moisture, perspiration or saliva may be pipetted onto working electrodes 202-206. In at least one embodiment, multiple working electrodes may be present on electrochemical biosensor 200 to simultaneously detect more than one target analyte.

FIG. 2B illustrates a lower plan view of electrochemical bio sensor 200, in accordance with at least one embodiment. In at least one embodiment, lower surface 215 of electrochemical biosensor 200 comprises microheater 218. In at least one embodiment, microheater 218 comprise a meandered conductive trace. In at least one embodiment, microheater 218 may comprise copper, aluminum, glassy carbon, carbon nanotubes or carbon dots. In at least one embodiment, microheater 218 is formed on lower surface 215 by thin film patterning methods, such as screen printing, vacuum sputtering or evaporation.

In at least one embodiment, microheater 218 is coupled to interconnect pads 220 by traces 222. In at least one embodiment, interconnect pads 220 and traces 222 may comprise the same material as does microheater 218. In at least one embodiment, interconnect pads 220 and traces 222 may be formed on lower surface 215 by similar thin film deposition methods as described for microheater 218. In at least one embodiment, interconnect pads 220 may enable electrical coupling to microheater 218 by insertion of electrochemical biosensor 200 into a readout device connector as described above.

In at least one embodiment, current may be sent to microheater 218 through interconnect pads 220 and traces 222 to heat working electrodes (e.g., working electrodes 202-206) on opposing upper surface 201 to control the surface temperature of working electrodes. In at least one embodiment, the local temperature of the working electrodes may affect the binding affinity of analytes within the MIP layer. In at least one embodiment, local heating of working electrodes may reduce the binding affinity of target analytes. In at least one embodiment, local heating of the working electrode may also cause the MIP layer to swell, favoring dissociation of the target analyte molecules from the shape-selective cavity binding sites within the MIP layer. In at least one embodiment, target analyte molecules may attach to shape-selective cavities within the MIP layer at room temperature (e.g., 20° C.-25° C.) and desorb when working electrodes are heated to 37° C. or above. In at least one embodiment, binding and dissociation of target analyte molecules may be repeatable, enabling time-dependent continuous detection of target analytes.

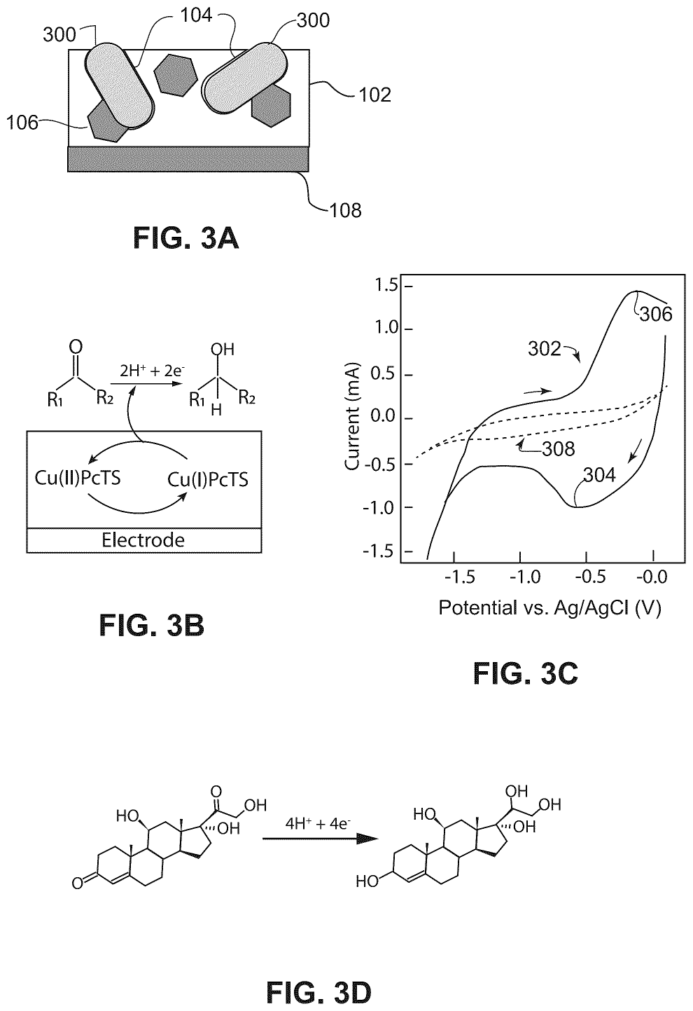

FIGS. 3A-3D illustrate a target analyte detection scheme using electrochemical biosensor 200, in accordance with at least one embodiment. In FIG. 3A. target analyte molecules 300 are shown bound to shape-selective cavities 104 within MIP layer 102. In at least one embodiment, target analyte molecules 300 may be within close proximity (e.g., 1 to 10 nm) of electrocatalytic centers 106 to undergo redox chemistry with electrocatalytic centers 106. For example, in at least one embodiment, electrocatalytic centers 106 may comprise copper(II) phthalocyanine tetrasulfonate (Cu(II)PcTS) or other suitable phthalocyanine derivatives, as an electrocatalyst that may react with the target analyte. In at least one embodiment, the target analyte may be cortisol, as shown (e.g., see FIG. 3D).

FIG. 3B illustrates a proposed reaction mechanism for cortisol reduction by electrocatalytic centers 106 comprising CuPcTS, in accordance with at least one embodiment. In at least one embodiment, four electrons may be supplied by electrode 108 (e.g., any one of working electrodes 202-206) per molecule of cortisol. The copper metal center in copper phthalocyanine (which resides in electrocatalyst centers 106), may undergo direct reversible reduction by electrode 108 from Cu(II) to Cu(I) or Cu ° (neutral copper).

While cortisol is referenced repeatedly as an exemplary target analyte in this disclosure, the disclosed electrochemical biosensor (e.g., electrochemical biosensor 200) may be employed to detect and measure a large variety of other substances, such as, but not limited to, molecules with phenol and thiol groups, steroid hormones (e.g., cortisol, progesterone, estradiol) and drugs (e.g., tetrahydrocannabinol, morphine, cocaine, codeine, methamphetamine) and their metabolites, neurotransmitters (e.g., dopamine, serotonin, norepinephrine, epinephrine), carbohydrates (e.g., glucose, fructose), ions (e.g., sulfide ion), antibiotics (e.g., tetracycline), alcohols, cholesterol, vitamin (e.g., ascorbic acid, vitamin B1), waste product (e.g., uric acid), amino acid (e.g., glycine, cysteine), antioxidant (N-acetylcysteine, glutathione), and some inorganic compounds (hydrazine, hydroxylamine).

As shown in FIG. 3B, the reaction may proceed by electron transfer in the form of a hydride (e.g., H⁻) transfer from the copper atom to the carbon atom of the keto group, with a simultaneous proton transfer from the solution (e.g., water) to the ketone oxygen atom. Two electrons are transferred between the copper atom and the keto group. In this example, cortisol is converted to tetrahydrocortisol. As noted earlier, the copper atom may cycle between Cu⁺² and Cu⁰ in one step or between Cu⁺² and Cu⁺¹ and Cu⁺¹ and Cu⁰ in two steps. Cyclic voltammogram (CV) 302 shown in FIG. 3C show reduction peak 304 and oxidation peak 306 of the copper center occurring at about −0.6V and −0.1V vs. Ag/AgCl, respectively. The CV shown in the figure is of the catalytic film comprising the MIP and CuPcTS (EC-P). In the same figure, CV 308 of pure PPy polymer is superimposed to show the lack of electrochemical activity of the pure polymer.

FIG. 3D illustrates structural views of cortisol and tetrahydrocortisol, which is a reduction product of cortisol. Keto groups at the C3 and C20 carbon positions are reduced to hydroxyl groups at those same positions, in accordance with at least one embodiment. As shown, four electrons and protons are involved in the redox reaction.

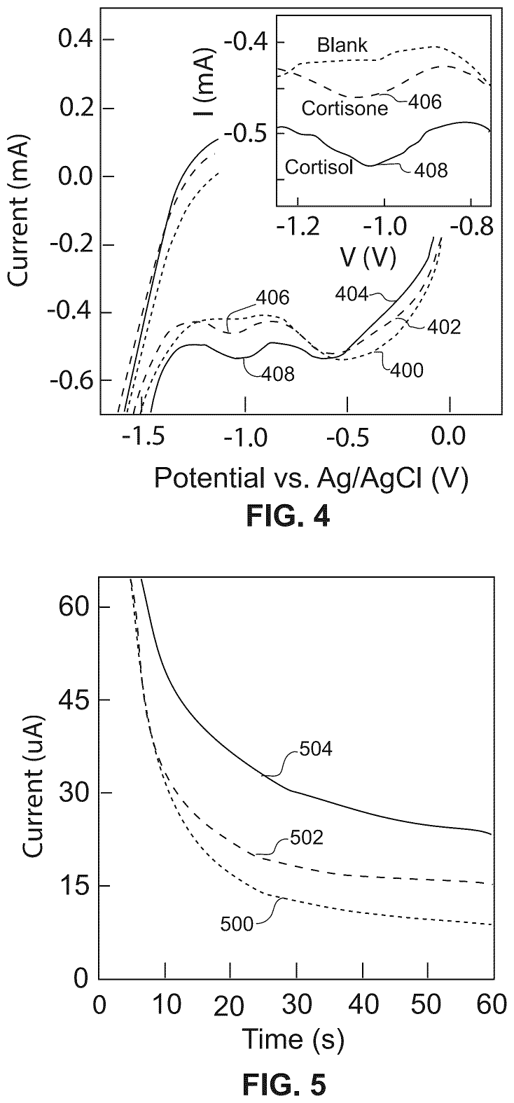

FIG. 4 illustrates a family of cyclic voltammograms comparing cyclic voltammograms of an EC-P-coated working electrode in the presence of blank electrotype, electrolyte containing 1 uM (micromolar) cortisone and electrolyte containing 1 uM cortisol, in accordance with at least one embodiment. In at least one embodiment, the working electrode comprises an EC-P layer. In at least one embodiment, an EC-P layer may comprise a polypyrrole (PPy) layer having no shape-selective cavities (e.g., shape-selective cavities 104), but comprising electrocatalytic centers (e.g., electrocatalytic centers 106). In at least one embodiment, CV 400 (stippled curve), CV 402 (dashed curve) and CV 404 (solid curve) are respectively shown for a for a blank electrolyte (e.g., a phosphate buffer solution at pH 7.4), a similar electrolyte comprising 1 uM cortisone, and a similar electrolyte comprising 1 uM cortisol. Cortisone is structurally similar to cortisol but is distinguished from cortisol in that it has a ketone oxygen at position C11, whereas cortisol has a hydroxyl group at position C11.

In at least one embodiment, reduction peaks 406 and 408 occur at approximately −1.0V vs. Ag/AgCl on CV 402 and CV 404 for cortisone and cortisol, respectively. An enlargement of reduction peaks 406 and 408 are shown in the inset. A CuPcTS reduction peak is also shifted from approximately −0.5V in blank electrolyte to approximately −0.6V in the presence of cortisone and cortisol.

FIG. 5 illustrates a family of chronoamperometric curves comparing chronoamperometric responses of an EC-P coated working electrode in the presence of cortisone and cortisol as target analytes, in accordance with at least one embodiment. In at least one embodiment, comparison is made to the response of a working electrode coated with a PPy polymer film (e.g., EC-P film) but having no shape-selective cavities present. In at least one embodiment, chronoamperogram 500 (stippled curve), chronoamperogram 502 (dashed curve) and chronoamperogram 504 (solid curve) respectively show electrochemical responses for an EC-P working electrode in the presence of blank electrolyte (e.g., phosphate buffer at pH 7.4), electrolyte containing 1 uM cortisone and electrolyte containing 1 uM cortisol.

In at least one embodiment, chronoamperograms 500, 502 and 504 are responses of the working electrode in the test electrolytes at a constant potential of −1V vs. Ag/AgCl stepped from a more anodic potential. Electrode response illustrated by chronoamperogram 504 for 1 uM cortisol is the greatest in comparison to chronoamperogram 502 and chronoamperogram 500 for 1 uM cortisone and blank electrolyte respectively, in accordance with CV reduction peak comparisons shown in FIG. 4. As will be described below, the current measured at a standardized time as determined by similar chronoamperograms may be correlated to solution (e.g., electrolyte) concentrations of cortisol as a target analyte.

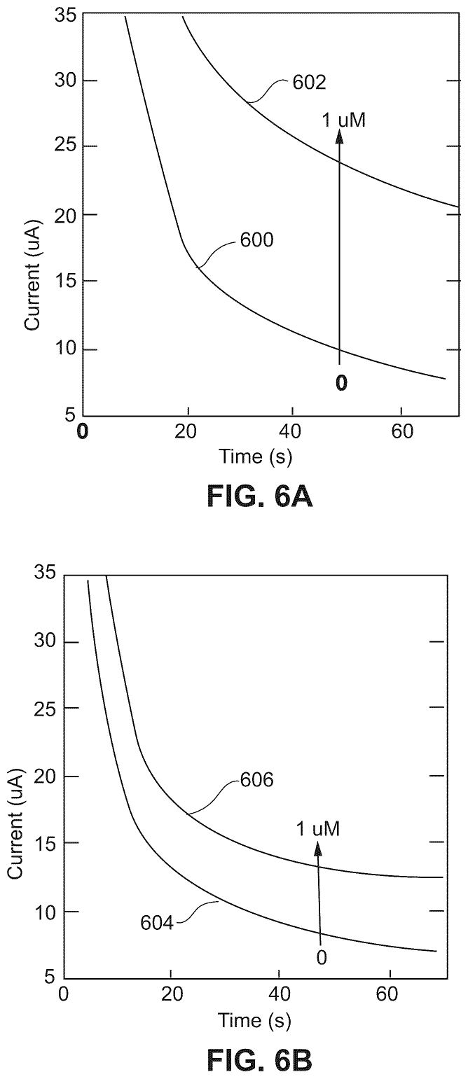

FIG. 6A illustrates a progression of chronoamperometry response curves for a working electrode coated by an EC-MIP film comprising cortisol binding sites, in accordance with at least one embodiment. In at least one embodiment, curve 600 and curve 602 are measures of chronoamperometric responses of a working electrode (e.g., any of working electrodes 202-206) coated by a MIP layer doped with electrocatalyst (e.g., MIP layer 102 doped by electrocatalytic centers 106). In at least one embodiment, the MIP layer comprises specific binding sites (e.g., shape-selective cavities 104) for cortisol. Curve 600 shows an exemplary chronoamperometric response of such a working electrode in an electrolyte with 0 uM cortisol (e.g., no cortisol present). In at least one embodiment, curve 602 shows an exemplary chronoamperometric response of the same working electrode in an electrolyte comprising 1 uM cortisol. In at least one embodiment, the electrolyte comprises a phosphate buffer at pH 7.4. In at least one embodiment, curves 600 and 602 are measured at −1V vs. Ag/AgCl. While curves 600 and 602 are shown in FIG. 6A for 0 uM and 1 uM, respectively, a progression of chronoamperometric curves measured at intermediate cortisol concentrations between 0 uM and 1 uM may be fitted between curves 600 and 602.

FIG. 6B illustrates a progression of chronoamperometry response curves for a working electrode coated by an EC-P film comprising polymer without binding sites, in accordance with at least one embodiment. In at least one embodiment, curve 604 and curve 606 are measures of chronoamperometric responses of a working electrode (e.g., any of working electrodes 202-206) coated by a non-MIP polymer layer doped with electrocatalyst. In at least one embodiment, the non-MIP layer has no specific binding sites. In at least one embodiment, curve 604 shows an exemplary chronoamperometric response of such a working electrode in an electrolyte with 0 uM cortisol (e.g., no cortisol present). In at least one embodiment, curve 606 shows an exemplary chronoamperometric response of the same working electrode in an electrolyte comprising 1 uM cortisol. In at least one embodiment, the electrolyte comprises a phosphate buffer at pH 7.4. In at least one embodiment, curves 604 and 606 are measured at −1V vs. Ag/AgCl. While curves 604 and 606 are shown in FIG. 6B for 0 uM and 1 uM, respectively, a progression of chronoamperometric curves measured at intermediate cortisol concentrations between 0 uM and 1 uM may be fitted between curves 604 and 606, in accordance with at least one embodiment.

Curves 604 and 606 may be compared to curves 600 and 602 in FIG. 6A, respectively, for comparison of electrochemical responses of EC-P films and EC-MIP films, in accordance with at least one embodiment. In at least one embodiment, an EC-P film is an electrocatalytically doped polymer film (e.g., PPy) but having no molecular imprinted shape-specific cavities for target analyte molecules. In at least one embodiment, an EC-MIP film is an electrocatalytically doped polymer film comprising molecular imprinted shape-specific cavities, (e.g., shape-specific cavities 104), such as MIP layer 102. In at least one embodiment, for EC-P film coated working electrodes, no specific binding sites are available, thus cortisol may non-specifically adsorb onto the EC-P film. In at least one embodiment, the response of the working electrode comprising an EC-MIP film is significantly greater than the response of a working electrode comprising an EC-P film (e.g., by comparison of curve 606 in FIG. 6B to curve 602 in FIG. 6A). In at least one embodiment, a working electrode coated by an EC-MIP layer, such as any of working electrodes 202-206, for example, has a significantly greater sensitivity to a target analyte than a similar electrode coated by an EC-P layer.

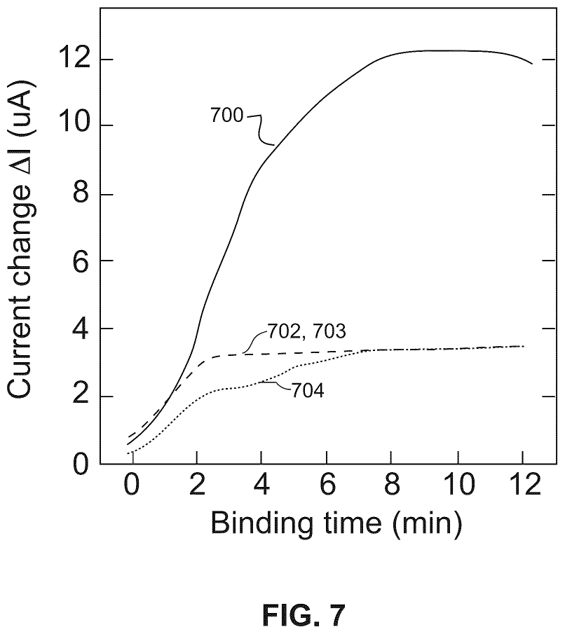

FIG. 7 illustrates a binding isotherm for cortisol and cortisone on EC-MIP coated working electrodes and EC-P coated working electrodes, in accordance with at least one embodiment. In at least one embodiment, curve 700 is a measure of an electrochemical current of an EC-MIP coated working electrode in the presence of 100 nM (nanomolar) cortisol relative to a current measured in a blank sample containing no target analytes. In at least one embodiment, to reduce the influence of non-specific adsorption of target analyte on the catalytic film, a differential current measurement method is employed. In at least one embodiment, an electrochemical current for a target analyte on a first working electrode comprising an EC-MIP catalytic layer is recorded. In at least one embodiment, subsequently or simultaneously a second working electrode comprising a non-MIP EC-P catalytic film (e.g., no imprinted cavities) from the same analyte solution. Both working electrodes may be on the same biosensor, for example. In at least one embodiment, a difference of the two electrochemical currents measured at the same electrochemical potential may be computed. In at least one embodiment, differential current may be calibrated to the concentration of target analyte.

In at least one embodiment, differential current (ΔI) displayed is a difference between current measured in the presence of 100 nM cortisol and a blank electrolyte, after an induction period of 40 seconds when the electrode is stepped to 1V vs. Ag/AgCl from an anodic potential. In at least one embodiment, the working electrodes are exposed to the electrolyte for different time periods (in minutes) prior to measurement. In at least one embodiment, exposure allows analytes to undergo adsorption and binding to the EC-MIP film or the EC-P film.

In at least one embodiment, curve 702 is a measure of the response of the same working electrode in the presence of 100 nM cortisone. In at least one embodiment, a third measurement curve 703 representing an EC-P working electrode in the presence of 100 nM cortisol is present in FIG. 7, but substantially indistinguishable from curve 702 and thus not readily evident, Curve 703 is again indicative of the selective sensitivity amplification due to molecularly imprinted shape-specific cavity binding sites in MIP films (e.g., MIP layer 102) relative to non-MIP films. In at least one embodiment, the non-specific binding of target analytes on a non-MIP film may not enable target analyte molecules (e.g., cortisol) to be distinguished in mixtures of two or more related substances. In at least one embodiment, such mixtures may comprise a target analyte such as cortisol mixed with background levels of cortisone and other corticosteroids in bodily fluid samples, for example. In at least one embodiment, curves 702 and 703 also indicate that molecular-imprinted shape-specific cavity binding sites (e.g., shape-specific cavities 104) may significantly exclude non-identical molecules having minor structural differences from the target molecule, such as minor structural differences between cortisone and cortisol.

In at least one embodiment, curve 704 is a measure of 1 nM cortisol on the EC-MIP coated working electrode. In at least one embodiment, all curves 700, 702 and 704 reach a plateau after several minutes, depending on the type of film. For example, curves 702 and 703, representing non-specific binding may reach a plateau after approximately two minutes, while curves 700 and 704, representing specific binding, may reach a plateau after six to seven minutes. Such plateaus may indicate that a binding equilibrium is reached. In at least one embodiment, a faster rise to equilibrium may indicate more rapid adsorption/binding kinetics. As equilibrium may be reached more slowly for the EC-MIP film than for an EC-P film, target molecules may take longer to bind with shape-specific cavities than to non-specifically adsorb on a polymer film, in accordance with at least one embodiment. In at least one embodiment, cortisol binding kinetics on both EC-P films and EC-MIP films may follow a Langmuir-Freundlich isotherm.

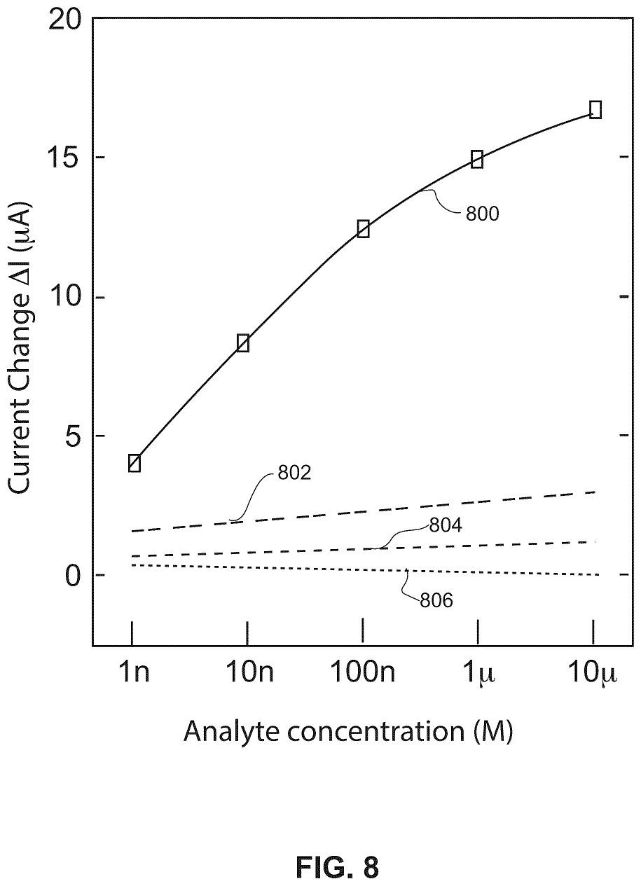

FIG. 8 illustrates binding isotherms of cortisol and other analytes on an EC-MIP film-coated working electrode, in accordance with at least one embodiment. In at least one embodiment, curve 800 (solid curve) is a response curve for an EC-MIP coated working electrode (e.g., any one of working electrodes 202, 204 and 206) measured in the presence of different concentrations of cortisol in a phosphate buffer electrolyte (e.g., pH 7.4). In at least one embodiment, current is a differential current (ΔI) with respect to a blank electrolyte containing no analytes. Curve 800 shows a substantially linear portion between 1 nM and 100 nM cortisol. Beyond cortisol concentrations of 100 nM, curve 800 enters a non-linear region and begins to reach a plateau. Based on curve 800, in at least one embodiment, an electrochemical biosensor such as electrochemical biosensor 200 employing working electrodes 202-206 may have good cortisol measurement linearity across four orders of magnitude between 100 pM (picomolar) and 1 uM.

Curves 802-806 in FIG. 8 show responses to potentially interfering model substances progesterone, glucose and lactic acid, respectively, that may coexist with cortisol in some bodily fluid samples. In at least one embodiment, curve 802 (alternating dashed curve) is a response curve for the same or similar EC-MIP working electrode in the presence of progesterone, a corticosteroid structurally related to cortisol. In at least one embodiment, curve 802 is substantially below curve 800, indicative of non-specific binding. In at least one embodiment, curve 804 (dashed curve) is a response curve for the same or similar EC-MIP working electrode in the presence of glucose, a molecule structurally and chemically unrelated to cortisol. In at least one embodiment, curve 804 is substantially below curve 800 and curve 802, also indicative of a low binding affinity for the EC-MIP layer. In at least one embodiment, curve 806 (stippled curve) is a response curve for the same or similar EC-MIP working electrode in the presence of lactic acid, a small molecule both structurally and chemically unrelated to cortisol. Curve 806 is further suppressed in comparison to curves 800, 802, and 804, indicative of very small binding affinity for the EC-MIP layer. In at least one embodiment, these three model substances may not significantly interfere with cortisol due to their low binding affinities for the EC-MIP films.

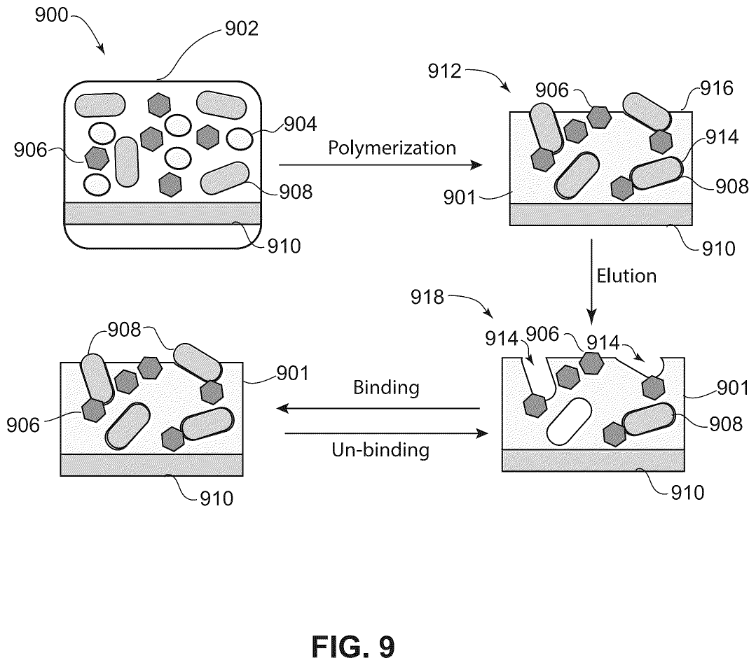

FIG. 9 illustrates an exemplary process flow of a method of producing the disclosed electrochemical biosensor (e.g., electrochemical biosensor 200), in accordance with at least one embodiment. In at least one embodiment, film precursor 900 comprises liquid matrix 902, comprising a mixture of polymer precursor 904 (e.g., monomers), electrocatalyic centers 906 (e.g., CuPcTS) and target analyte molecules 908. In at least one embodiment, film precursor 900 may be deposited on electrode 910 (e.g., similar to electrode 108).

Electrode(s) 910 may be screen printed onto an electrically insulative support substrate (e.g., substrate 208). In at least one embodiment, substrate may comprise a polymer such as polyethylene terephthalate (PET). In at least one embodiment, insulative materials may be employed as substrates, such as glass, quartz, undoped silicon wafer or oxidized silicon wafer and ceramic materials such as alumina, titania, etc. In at least one embodiment, screen printed electrodes may comprise films comprising glassy carbon, basal pyrolytic carbon, graphene, carbon nanotubes and noble metals such as platinum, gold or silver in the form of nanoparticles embedded within carbon matrices or as films of the pure metals. In at least one embodiment, suitable electrode materials may include indium tin oxide (ITO).

In at least one embodiment, electrode(s) 910 may be screen printed on a substrate. In at least one embodiment, a carbon ink may be printed on to a PET substrate. In at least one embodiment, carbon ink may be patterned by the print design to include conductive electrode bases, traces (e.g., traces 216) and interconnect pads (e.g., interconnect pads 214). In at least one embodiment, while a carbon ink may be employed, other suspensions comprising metallic nanoparticles and microparticles such as gold, platinum, silver, etc., may also be employed. In at least one embodiment, screen printed structures may range from several microns up to several hundred microns in thickness.

In at least one embodiment, other thin film techniques may be employed to deposit conductive structures. In at least one embodiment, vacuum sputtering or evaporation may be employed to deposit metal films. In at least one embodiment, deposited structures may range in thickness from several nanometers to 1 or 2 microns with low resistivities. In at least one embodiment, a reference electrode (e.g., reference electrode 210) may be made by printing an Ag/AgCl ink followed by a bake at 110° C. for 10 minutes.

In at least one embodiment, MIP layer 912 (e.g., an EC-MIP film) may be polymerized on electrode 910. In at least one embodiment, MIP film precursor 900 may be electropolymerized to form MIP layer 912. In at least one embodiment, electropolymerization of MIP film precursor 900 may be performed by cyclic voltammetry, where the electrode potential is swept multiple times between two terminal potentials. In at least one embodiment, potential may be swept ten times between 0V and +0.95V vs. Ag/AgCl in a solution containing pyrrole monomer. In at least one embodiment, MIP film precursor may comprise target analyte (e.g., cortisol) in low concentration (e.g., 10 mM) for cavity imprint, and electrocatalytic centers 906 (e.g., CuPcTS at 50 mM).

In at least one embodiment, a stratified catalytic film is synthesized, whereby molecules of the electrocatalyst are first attached to the electrode surface by physisorption or chemisorption. In at least one embodiment, the MIP layer may be grown over the catalytic layer, forming electrocatalytic film 150, for example. In at least one embodiment, an insulative layer comprising polymer matrix alone is grown over the MIP layer by methods described above, forming electrocatalytic films 120 or 160, for example.

In at least one embodiment, the MIP layer 912 may be formed by thermal polymerization or photopolymerization of MIP film precursor 900 directly on the surface of electrode 910. MIP layer 912 may be grown to thicknesses ranging between 5 nm to 1 micron. In at least one embodiment, film thickness may be limited to 200 nm or less for optimal mass transfer of target analyte and electrolyte penetration into the catalytic film.

In at least one embodiment, a single homogeneous catalytic film is synthesized as MIP layer 912, whereby polymer precursor 904 (e.g., pyrrole monomer) is polymerized to a solid polymer (e.g., PPy by electropolymerization, photopolymerization or thermal polymerization). In at least one embodiment, target analyte molecules 908 and electrocatalytic centers 906 are immobilized within the polymer matrix 901 (e.g., PPy).

In at least one embodiment, shape-selective cavities 914 are formed around target analyte molecules 908 by exclusion of polymer in the spaces occupied by target analyte molecules 908. In at least one embodiment, shape-selective cavities 914 may intersect surface 916 of MIP layer 912. In at least one embodiment, shape-selective cavities 914 may be vacated by soaking MIP layer 912 in ethanol for 10 minutes while stirring, forming activated MIP layer 918. In at least one embodiment, activated MIP layer 918 comprises a plurality of empty shape-selective cavities 914 within polymer matrix 901.

In at least one embodiment, target analyte molecules 908 may be bound to activated MIP layer 918 by soaking activated MIP layer 918 in electrolyte comprising target analyte molecules 908, as described above. In at least one embodiment, target analyte molecules 908 may infiltrate polymer matrix 901, finding their way to shape-selective cavities to which they may have a high binding affinity.

In at least one embodiment, bound target analyte molecules 908 may be unbound (e.g., desorbed) from MIP layer 912 by thermal heating of MIP layer 912, whereby MIP layer 912 is reverted to activated MIP layer 918. In at least one embodiment, an integrated microheater (e.g., microheater 218) on the back of the electrochemical biosensor (e.g., electrochemical biosensor 200) is employed. In at least one embodiment, the MIP layer 912 may be renewed by heating the catalytic film to cause dissociation of bound target analyte molecules 908 from shape selective cavities 914. In at least one embodiment, heating operation may cause swelling of the MIP layer 912 and shape-selective cavities 914, while thermodynamically reducing the binding affinity of the analyte.

In at least one embodiment, the integrated microheater may be positioned opposite the working electrodes on the first side of the substrate. In at least one embodiment, integrated microheater may provide heat to desorb target analyte (e.g., cortisol) bound in shape-specific cavities 914. As MIP layer 912 may become saturated with target electrolyte during an individual assay, desorption of trapped analyte may be performed to renew the working electrode for subsequent assays, in accordance with at least one embodiment.

In at least one embodiment, heating of MIP layer 912 by the integrated microheater may cause the MIP layer to swell, opening shape-selective cavities 914. In at least one embodiment, raising the temperature of the MIP layer 912 may also decrease the binding equilibrium constant, enabling release of bound target analyte molecules 908.

In at least one embodiment, bound target analyte molecules 908 may be removed from shape-selective cavities 914 by voltage-induced polymer swelling. In at least one embodiment, voltage-induced swelling comprises inclusion of additional electrolytes into the catalytic film. In at least one embodiment, negatively or positively charged electrolyte ions or polyelectrolytes may be added to film precursor 900 for permanent incorporation of charged groups into the polymer matrix.

In at least one embodiment, polymer matrix 901 may contain monomers or charged polyelectrolytes anchored to or entangled within polymer matrix 901. In at least one embodiment, polymer matrix 901 may be doped with cationic species such as positively charged ammonium or amine groups. In at least one embodiment, polymer matrix 901 may be doped with anionic specie, such as charged sulfonic acid, carboxylic acid, or phosphoric acid groups. In at least one embodiment, these electrolytes may remain in the MIP layer 912 as a potential of like polarity may be applied to the electrode, attracting counterions into the film. Counterions may also bring associated water molecules. In at least one embodiment, MIP layer 912 may be caused to swell by a flow of water and counterions into polymer matrix 901. In at least one embodiment, the shape affinity of shape-selective cavities 914 may be reduced, allowing bound target analyte molecules 908 to escape MIP layer 912.

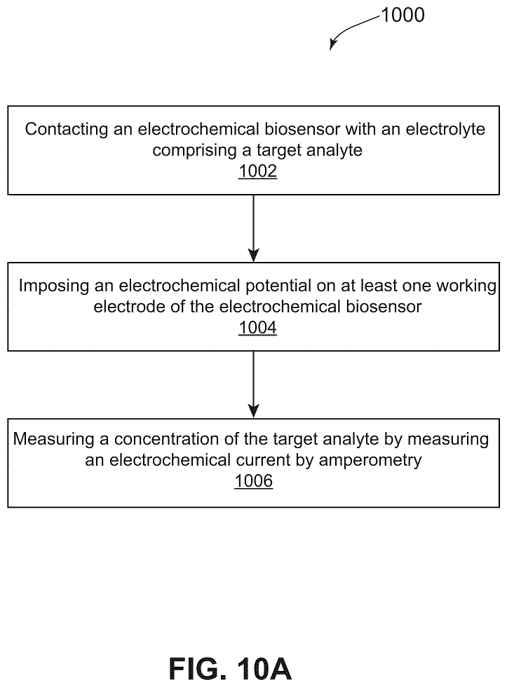

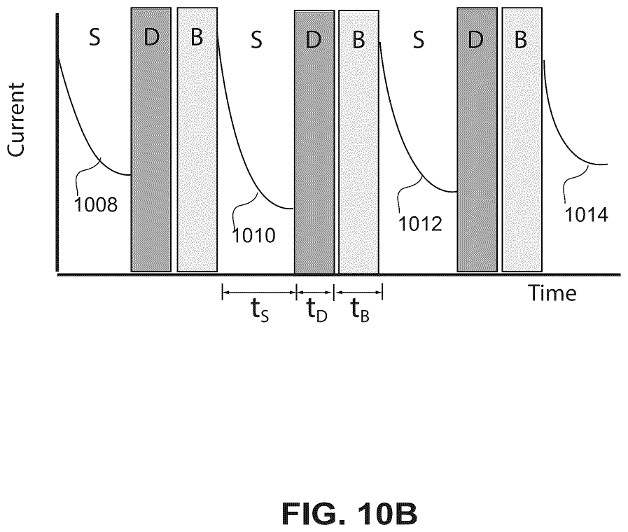

FIG. 10A illustrates process flowchart 1000 summarizing an exemplary method for using an electrochemical biosensor in a biological assay, in accordance with at least one embodiment. At operation 1002, an electrochemical biosensor according to at least one embodiment, such as disclosed electrochemical biosensor 200, is contacted with a sample solution comprising a bodily fluid sample, obtained from a subject or patient. In at least one embodiment, the bodily fluid sample may be a neat sample measured directly, or may be diluted in a prepared aqueous electrolyte solution. In at least one embodiment, a prepared electrolyte may comprise a pH buffer, such as a phosphate buffer solution buffered at a suitable pH (e.g., pH 7). In at least one embodiment, electrolyte solution may comprise a target analyte, such as cortisol, in micromolar or nanomolar concentrations. In at least one embodiment, target analyte molecules may selectively bind to an MIP layer on one or more working electrodes on the electrochemical biosensor. In at least one embodiment, binding affinity for the target analyte may be several times greater than for chemical analogs of the target analyte. In at least one embodiment, cortisol may bind to the MIP layer with at least four times the rate of cortisone binding.

In at least one embodiment, multiple working electrodes may be present on the electrochemical biosensor (e.g., working electrodes 202, 204, and 206). In at least one embodiment, each working electrode may be coated with a different MIP for assaying different target analytes contained in the same bodily fluid sample.

In at least one embodiment, the electrochemical biosensor may be dipped into a bath containing the electrolyte such that the integrated electrochemical cell is immersed. In at least one embodiment, electrolyte may be pipetted over the electrochemical biosensor, flooding the electrodes, including the one or more working electrodes, the reference electrode and the counter electrode.

At operation 1004, integrated interconnect pads on the electrochemical biosensor (e.g., interconnect pads 214) may be connected to an interface of a biosensor reader (e.g. reader 1204, described below). In at least one embodiment, biosensor may be plugged into a slotted connector on the biosensor reader. In at least one embodiment, a multi-analyte biosensor may have separately regulated working electrodes for different target analytes. In at least one embodiment, the biosensor reader may comprise a potentiostat circuit that is operable to control one or more potentials on the one or more working electrodes.

At operation 1006, one or more electrochemical currents generated at the one or more working electrodes may be measured and recorded. In at least one embodiment, electrochemical currents may be measured by an amperometric method, such as chronoamperometry, whereby the working electrode potential is stepped from an idle potential to an active reduction (or oxidation) potential, for example at −1V vs. Ag/AgCl, and the resulting electrochemical current is measured at a quasi-steady state value after an initial period of current decay. In at least one embodiment, quasi-steady state period may be determined by the rate of decay after a number of decay time constants. In at least one embodiment, electrochemical current is related to the concentration of target analyte in the electrolyte solution. In at least one embodiment, a calibration curve or look-up table may be consulted to determine the concentration of target analyte contained by the electrolyte solution, and then by back-calculation, the concentration of target analyte may be determined for the undiluted bodily fluid sample and therefore the circulating target analyte level in the body of the subject or patient.

FIG. 10B illustrates an exemplary purge/renewal process by dissociation of the bound target analyte molecules, in accordance with at least one embodiment. In at least one embodiment, the disclosed electrochemical biosensor may be employed for continuous monitoring of bodily fluids. In at least one embodiment, a repetitive sequence comprising a sensing operation, a dissociation operation and a binding operation may be employed to electrochemically detect and measure an analyte by selectively binding the analyte within the MIP layer. In at least one embodiment, repetitive use of the disclosed electrochemical biosensor may be advantageous for continuous monitoring of a bodily fluid such as whole blood, plasma, saliva, sweat, or urine. In at least one embodiment, disclosed electrochemical biosensor may be employed as a portable or wearable device.

In at least one embodiment, FIG. 10B shows a method to enable continuous detection of target analytes. In at least one embodiment, continuous detection may be performed by an operational sequence of sensing (S), dissociation (D) and binding (B). Time periods for each phase are indicated by durations denoted by t_{S}, t_{D}, and t_{B}. In at least one embodiment, sensing phases are indicated by chronoamperometric curves 1008, 1010, 1012, and 1014. In at least one embodiment, duration t_{S} may last one minute or less. A working electrode (e.g., any of working electrodes 202-206) may be potentiostated at an active reduction potential of −1V vs. Ag/AgCl, for example. In at least one embodiment, current may be measured after an induction period (e.g., 40 seconds). In the dissociation phase (D), a heating pulse for thermal dissociation, or an electrochemical pulse for voltage-induced dissociation as described above (e.g., see FIG. 9) by be performed for duration t_{D}. During t_{D}, target analyte molecules are dissociated from shape-selective cavities within and EC-MIP film. In at least one embodiment, t_{D} may be five minutes or less. In at least one embodiment, during the binding phase (B), the MIP layer on the working electrode is exposed to analyte-containing electrolyte to bind target analyte from a new sample for duration t_{B}, for example. In at least one embodiment, t B may be 10 minutes or less.

In at least one embodiment, a patient may employ the disclosed electrochemical biosensor (e.g., electrochemical biosensor 200) as a point of care medical device for automatic continuous or intermittent monitoring of a biologic analyte, such as cortisol levels in blood. In at least one embodiment, electrochemical biosensor may also be part of a wearable medical device for portable point of care use. For repetitive monitoring, the analyte may be purged from the MIP layer, thus renewing the MIP layer for a subsequent measurement cycle.

FIG. 11 illustrates a chemical structure of copper phthalocyanine tetrasulfonate (CuPcTS), in accordance with at least one embodiment. In at least one embodiment, CuPcTS is an electrocatalytic center, as described above.

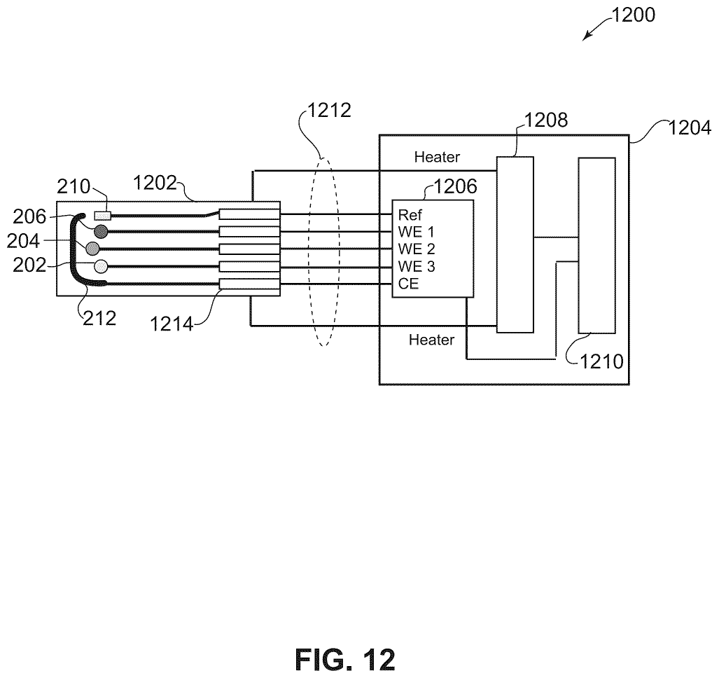

FIG. 12 illustrates a block diagram of system 1200, in accordance with at least one embodiment. In at least one embodiment, system 1200 comprises electrochemical biosensor 1202 electrically coupled to reader 1204 in accordance with at least one embodiment. In at least one embodiment, reader 1204 comprises potentiostat circuitry 1206, temperature controller circuitry 1208 and processor 1210. In at least one embodiment, reader 1204 comprises interface 1212. In at least one embodiment, interface 1212 comprises a plurality of contacts that may mate to interconnect pads 1214 on an electrochemical biosensor 1202. In at least one embodiment, interface 1212 may be electrically coupled to cables carrying voltages to electrodes on electrochemical biosensor 1202 mated to interface 1212. For example, electrochemical biosensor 1202 may insert into interface 1212, whereby interconnect pads 1214 may be contacted to spring-loaded interconnects within an insertion slot in interface 1212. In at least one embodiment, interface 1212 may be electrically coupled to one or more cables extending from potentiostat circuitry 1206. Such cabling may comprise wires coupling potentiostat circuitry 1206 to one or more working electrodes (e.g., working electrodes 202-206) integrated on the electrochemical biosensor 1202, as well as to an integrated reference electrode (e.g., reference electrode 210) and an integrated counter electrode (e.g., counter electrode 212).

In at least one embodiment, during operation of electrochemical biosensor 1202, potentiostat circuitry 1206 may maintain a potential on the one or more working electrodes 202-206 with respect to reference electrode 210. In at least one embodiment, potentiostat circuitry 1206 may maintain a steady potential (e.g., voltage), or may scan or sweep electrode potentials. In at least one embodiment, working electrodes 202-206 may be independently potentiostated with respect to reference electrode 210 for independent assays of different analytes within a sample on a single electrochemical sensor, for example.

In at least one embodiment, potentiostat circuitry 1206 may be electronically interfaced to processor 1210. In at least one embodiment, processor 1210 may be operable to execute software to control potentiostat circuitry 1206. For example, in at least one embodiment, processor 1210 may execute software to initiate chronoamperometric measurements or cyclic voltammograms. In at least one embodiment, processor 1210 may be operable to execute software routines to correlate data from measured chronoamperometric currents (e.g., as shown in FIG. 5 or FIG. 6A) to calibrated look up tables, yielding analyte concentration values for bioassays. In at least one embodiment, processor 1210 may be coupled to a memory (not shown) to store assay data.

In at least one embodiment, processor 1210 may be operable to perform electrode cycling operations for repeated measurements. In at least one embodiment, processor 1210 may command potentiostat circuitry 1206 to perform purge cycles to renew the MIP layers on working electrodes 202-206 by stepping the electrode potentials according to a protocol similar to the protocol shown in FIG. 10B.

In at least one embodiment, interface 1212 may couple temperature controller circuitry 1208 to an integrated microheater on electrochemical biosensor 1202 (e.g., microheater 218 on verso side of electrochemical biosensor 1202; not shown in FIG. 12). In at least one embodiment, cabled wires from temperature controller circuitry 1208 may supply electric current to the integrated microheater. In at least one embodiment, temperature controller circuitry 1208 may be electrically coupled to processor 1210. In at least one embodiment, processor 1210 may be operable to operate temperature controller circuitry 1208. For example, processor 1210 may be operable to execute software to set a temperature of working electrodes 202-206. In at least one embodiment, a temperature sensor may be integrated onto electrochemical biosensor 1202 and coupled to temperature controller circuitry 1208, or contacted to electrochemical biosensor 1202 through interface 1212.

The following examples are provided that illustrate at least one embodiment. An example can be combined with any other example. As such, at least one embodiment can be combined with at least another embodiment without changing the scope of the disclosure.

Example 1 is an electrochemical biosensor comprising an electrically insulative substrate; at least one working electrode on the electrolytically insulative substrate; and an electrocatalytic film on an electrochemically active surface of the working electrode, wherein the electrocatalytic film comprises a molecularly imprinted polymer (MIP) layer, wherein the MIP layer comprises a plurality of shape-selective cavities, and wherein the electrocatalytic film comprises a plurality of electrocatalytic centers.

Example 2 is the electrochemical biosensor of any example herein, particularly example 1, wherein the plurality of electrocatalytic centers comprises a non-biological catalyst.

Example 3 is the is the electrochemical biosensor of any example herein, particularly example 2, wherein the non-biological catalyst comprises a phthalocyanine ring comprising a metal atom; a single-atom catalyst comprising any one of copper, manganese, iron; cobalt, nickel, zinc, magnesium, aluminum or ruthenium; or a metallocene comprising any one of iron, cobalt, nickel, zinc, magnesium, titanium, vanadium, zirconium, hafnium, chromium, molybdenum, tungsten, manganese, ruthenium, osmium, or rhodium; or a compound comprising one of pendant nitroxide radical or pendant nitronyl nitroxide radical; or a metal-organic framework (MOF) comprising any one of copper, manganese, iron; cobalt, nickel, zinc, magnesium, aluminum, or ruthenium.

Example 4 is the electrochemical biosensor of any example herein, particularly example 3, wherein the metal atom is a copper atom.

Example 5 is the electrochemical biosensor of any example herein, particularly example 3, wherein the phthalocyanine ring comprises an ionizable sidechain, and wherein the ionizable sidechain comprises any of a sulphonate group, a phosphate group, a phosphonate group, a carboxylic acid group, or an amino group.

Example 6 is the electrochemical biosensor of any example herein, particularly example 1, wherein the electrocatalytic film comprises a single layer, and wherein the single layer comprises the MIP layer and the plurality of electrocatalytic centers immobilized within the MIP.

Example 7 is the electrochemical biosensor of any example herein, particularly example 6, wherein the single layer is a first layer comprising the MIP layer, and wherein the electrocatalytic film comprises a second layer over the first layer, wherein the second layer comprises a non-electrocatalytic material.

Example 8 is the electrochemical biosensor of any example herein, particularly example 1, wherein the electrocatalytic film comprises two or more layers, wherein the two or more layers comprise a first layer adjacent to an electrochemically active surface of the working electrode, wherein the first layer comprises the plurality of electrocatalytic centers, and wherein a second layer is an MIP layer over the first layer, and wherein the second layer comprises the plurality of shape-selective cavities.

Example 9 is the electrochemical biosensor of any example herein, particularly example 8, wherein the electrocatalytic film comprises a third layer over the second layer, and wherein the third layer comprises a non-electrocatalytic material.

Example 10 is the electrochemical biosensor of any example herein, particularly example 1, wherein the at least one working electrode comprises any one of carbon, gold, silver, platinum, palladium or indium tin oxide.

Example 11 is the electrochemical biosensor of any example herein, particularly example 1, further comprising a microheater on the electrically insulative substrate.

Example 12 is the electrochemical biosensor of any example herein, particularly example 1, wherein the MIP layer comprises at least one monomer, wherein the at least one monomer is any one of pyrrole, phenol, aminophenol, aniline, phenylenediamine, scopoletin, 3,4-ethylenedioxythiophene, vinyl monomer, acrylate monomer.

Example 13 is a sensor system, comprising an electrochemical biosensor, wherein the electrochemical biosensor comprises an insulative substrate, wherein at least one working electrode, a reference electrode and a counter electrode integrated are on the insulative substrate, wherein an electrocatalytic film is on the at least one working electrode, wherein the electrocatalytic film comprises the molecularly imprinted polymer (MIP) layer, and wherein the catalytic film comprises a non-biological electrocatalyst immobilized within the electrocatalytic film; and a reader comprising an interface to be coupled to the electrochemical biosensor.

Example 14 is the sensor system of any example herein, particularly example 13, wherein the electrochemical biosensor comprises a plurality of interconnect pads integrated on the insulative substrate and electrically coupled to the at least one working electrode, the reference electrode and the counter electrode.

Example 15 is the sensor system of any example herein, particularly example 14, wherein the plurality of interconnect pads is electrically coupled to a microheater integrated on the insulative substrate.

Example 16 is the sensor system of any example herein, particularly example 15, wherein the reader the interface is to be coupled to the plurality of interconnect pads.

Example 17 is the sensor system of any example herein, particularly example 16, wherein the reader further comprises a potentiostat circuitry electrically coupled to the interface, a temperature controller circuitry electrically coupled to the interface, and a processor electrically coupled to the poteniostat circuitry and to the temperature controller circuitry.

Example 18 is a method for making an electrochemical biosensor assembly, the method comprising patterning an integrated electrochemical cell on an electrically insulative substrate, wherein the integrated electrochemical cell comprises at least one working electrode, a reference electrode and a counter electrode; and forming an electrocatalytic film on a surface of the at least one working electrode, wherein the electrocatalytic film comprises a plurality of electrocatalytic centers comprising a non-biological electrocatalyst, and a molecular template substance; forming shape-selective cavities within the electrocatalytic film, wherein the molecular template substance is removed from the catalytic film.

Example 19 is the method of any example herein, particularly example 18, wherein forming the electrocatalytic film comprises polymerizing a monomer solution comprising the molecular template substance and the plurality of electrocatalytic centers, and wherein the molecular template substance is removed to form a molecular imprint polymer (MIP) comprising a plurality of shape-selective cavities.

Example 20 is the method of any example herein, particularly example 18, wherein forming the electrocatalytic film comprises immobilizing the electrocatalytic centers on the surface of the at least one working electrode and polymerizing a monomer solution comprising the molecular template substance, wherein a first layer comprising the plurality of electrocatalytic centers is adjacent to the surface of the at least one working electrode and wherein a second layer comprising the molecular template substance is above the first layer, and wherein the molecular template substance is removed to form a molecular imprint polymer (MIP) comprising a plurality of shape-selective cavities in the second layer.

Example 21 is a method for using an electrochemical biosensor, comprising contacting an electrochemical biosensor with a sample solution comprising a target analyte, wherein the electrochemical biosensor comprises an electrically insulative substrate, wherein an electrochemical cell is integrated on the electrically insulative substrate, wherein the electrochemical cell comprises at least one working electrode, and wherein a molecular imprinted polymer (MIP) layer is on an a surface of the at least one working electrode, and wherein the target analyte selectively binds to a plurality of shape-selective cavities within the MIP; imposing an electrochemical potential on the working electrode; and measuring an electrochemical current, wherein the electrochemical current is calibrated to a concentration of the target analyte in the solution.

Example 22 is the method for using an electrochemical biosensor of any example herein, particularly example 21, wherein measuring the electrochemical current comprises measuring the electrochemical current by chronoamperometry, wherein the target analyte undergoes a redox reaction at the electrochemical potential, and wherein the electrochemical current is recorded after an induction period.

Example 23 is the method for using an electrochemical biosensor of any example herein, particularly example 22, wherein measuring the electrochemical current by chronoamperometry comprises measuring a first electrochemical current on a first working electrode on the electrochemical biosensor, wherein the first working electrode comprises an MIP layer, wherein a second electrochemical current is measured from a second working electrode on the electrochemical biosensor, wherein the second working electrode comprises a non-specific polymer, wherein a differential current is computed by taking a difference between the first electrochemical current and the second electrochemical current, and wherein the differential current is calibrated to the concentration of the target analyte within the sample solution.

Example 24 is the method for using an electrochemical biosensor of any example herein, particularly example 21, further comprising desorbing the target analyte from the MIP layer, wherein desorbing the target analyte from the MIP layer comprises heating the first working electrode to desorb the target analyte from the MIP, wherein the electrochemical biosensor comprises a microheater integrated on the electrically insulative substrate.

Example 25 is the method for using an electrochemical biosensor of any example herein, particularly example 24, wherein desorbing the target analyte from the MIP layer comprises applying the electrochemical potential to the first working electrode to cause the MIP layer to swell.

Example 26 is the method for using an electrochemical biosensor of any example herein, particularly example 24, wherein desorbing the target analyte from the MIP layer comprises applying the electrochemical potential to the at least one working electrode to move the target analyte using an electric field.

Besides what is described herein, various modifications may be made to at least one embodiment thereof without departing from their scope. Therefore, illustrations of at least one embodiment herein should be construed as examples only, and not restrictive to the scope of the present disclosure.

We claim:

1. An electrochemical biosensor, comprising:
at least one working electrode on an electrically insulative substrate; and
an electrocatalytic film on a surface of the at least one working electrode, wherein the electrocatalytic film comprises a molecularly imprinted polymer (MIP) layer, wherein the MIP layer comprises a plurality of shape-selective cavities, and wherein the electrocatalytic film comprises a plurality of electrocatalytic centers.

2. The electrochemical biosensor of claim 1, wherein the plurality of electrocatalytic centers comprises a non-biological catalyst.

3. The electrochemical biosensor of claim 2, wherein the non-biological catalyst comprises:
a phthalocyanine ring comprising a metal atom;
a single-atom catalyst comprising any one of copper, manganese, iron; cobalt, nickel, zinc, magnesium, aluminum or ruthenium;
a metallocene comprising any one of iron, cobalt, nickel, zinc, magnesium, titanium, vanadium, zirconium, hafnium, chromium, molybdenum, tungsten, manganese, ruthenium, osmium, or rhodium; or
a compound comprising one of pendant nitroxide radical or pendant nitronyl nitroxide radical; or
a metal-organic framework (MOF) comprising any one of copper, manganese, iron;
cobalt, nickel, zinc, magnesium, aluminum, or ruthenium.

4. The electrochemical biosensor of claim 3, wherein the metal atom is a copper atom.

5. The electrochemical biosensor of claim 3, wherein the phthalocyanine ring comprises an ionizable sidechain, and wherein the ionizable sidechain comprises any of a sulphonate group, a phosphate group, a phosphonate group, a carboxylic acid group, or an amino group.

6. The electrochemical biosensor of claim 1, wherein the electrocatalytic film comprises a single layer, and wherein the single layer comprises the MIP layer and the plurality of electrocatalytic centers immobilized within the MIP layer.

7. The electrochemical biosensor of claim 6, wherein the single layer is a first layer comprising the MIP layer, and wherein the electrocatalytic film comprises a second layer over the first layer, wherein the second layer comprises a non-electrocatalytic material.

8. The electrochemical biosensor of claim 1, wherein the electrocatalytic film comprises two or more layers, wherein the two or more layers comprise a first layer adjacent to the surface of the at least one working electrode, wherein the first layer comprises the plurality of electrocatalytic centers, and wherein a second layer is an MIP layer over the first layer, and wherein the second layer comprises the plurality of shape-selective cavities.

9. The electrochemical biosensor of claim 8, wherein the electrocatalytic film comprises a third layer over the second layer, and wherein the third layer comprises a non-electrocatalytic material.

10. The electrochemical biosensor of claim 1, wherein the at least one working electrode comprises any one of carbon, gold, silver, platinum, palladium or indium tin oxide.

11. The electrochemical biosensor of claim 1, further comprising a microheater on the electrically insulative substrate.

12. The electrochemical biosensor of claim 1, wherein the MIP layer comprises at least one monomer, wherein the at least one monomer is any one of pyrrole, phenol, aminophenol, aniline, phenylenediamine, scopoletin, 3,4-ethylenedioxythiophene, vinyl monomer, or acrylate monomer.

13. A sensor system, comprising:
an electrochemical biosensor comprising an insulative substrate, wherein at least one working electrode, a reference electrode and a counter electrode are integrated on the insulative substrate, wherein an electrocatalytic film is on the at least one working electrode, wherein the electrocatalytic film comprises a molecular imprinted polymer (MIP) layer, and wherein the electrocatalytic film comprises a non-biological electrocatalyst immobilized within the electrocatalytic film; and
a reader comprising an interface to be coupled to the electrochemical biosensor.

14. The sensor system of claim 13, wherein the electrochemical biosensor comprises a plurality of interconnect pads integrated on the insulative substrate and electrically coupled to the at least one working electrode, the reference electrode and the counter electrode.

15. The sensor system of claim 14, wherein the plurality of interconnect pads is electrically coupled to a microheater integrated on the insulative substrate.

16. The sensor system of claim 15, wherein the interface is to be further coupled to the plurality of interconnect pads.

17. The sensor system of claim 16, wherein the reader further comprises a potentiostat circuitry electrically coupled to the interface, a temperature controller circuitry electrically coupled to the interface, and a processor electrically coupled to the potentiostat circuitry and to the temperature controller circuitry.

18. A method for making an electrochemical biosensor assembly, the method comprising:
patterning an integrated electrochemical cell on an electrically insulative substrate, wherein the integrated electrochemical cell comprises at least one working electrode, a reference electrode and a counter electrode;
forming an electrocatalytic film on a surface of the at least one working electrode, wherein the electrocatalytic film comprises a plurality of electrocatalytic centers comprising a non-biological electrocatalyst, and a molecular template substance; and
forming shape-selective cavities within the electrocatalytic film, wherein the molecular template substance is removed from the electrocatalytic film.

19. The method of claim 18, wherein forming the electrocatalytic film comprises polymerizing a monomer solution comprising the molecular template substance and the plurality of electrocatalytic centers, and wherein the molecular template substance is removed to form a molecular imprint polymer (MIP) comprising a plurality of shape-selective cavities.

20. The method of claim 18, wherein forming the electrocatalytic film comprises immobilizing the plurality of electrocatalytic centers on the surface of the at least one working electrode and polymerizing a monomer solution comprising the molecular template substance, wherein a first layer comprising the plurality of electrocatalytic centers is adjacent to the surface of the at least one working electrode and wherein a second layer comprising the molecular template substance is above the first layer, and wherein the molecular template substance is removed to form a molecular imprint polymer (MIP) comprising a plurality of shape-selective cavities in the second layer.

21. A method for using an electrochemical biosensor, comprising:
contacting the electrochemical biosensor with a sample solution comprising a target analyte, wherein the electrochemical biosensor comprises an electrically insulative substrate, wherein an electrochemical cell is integrated on the electrically insulative substrate, wherein the electrochemical cell comprises at least one working electrode, and wherein a molecular imprinted polymer (MIP) layer is on an a surface of the at least one working electrode, and wherein the target analyte selectively binds to a plurality of shape-selective cavities within the MIP layer;
imposing an electrochemical potential on the at least one working electrode; and
measuring an electrochemical current, wherein the electrochemical current is calibrated to a concentration of the target analyte in the sample solution.

22. The method of claim 21, wherein measuring the electrochemical current comprises measuring the electrochemical current by a chronoamperometric method, wherein the target analyte undergoes a redox reaction at the electrochemical potential, and wherein the electrochemical current is recorded after an induction period.

23. The method of claim 22, wherein measuring the electrochemical current by the chronoamperometric method comprises measuring a first electrochemical current on a first working electrode on the electrochemical biosensor, wherein the first working electrode comprises an MIP layer, wherein a second electrochemical current is measured from a second working electrode on the electrochemical biosensor, wherein the second working electrode comprises a non-specific polymer, wherein a differential current is computed by taking a difference between the first electrochemical current and the second electrochemical current, and wherein the differential current is calibrated to the concentration of the target analyte within the sample solution.

24. The method of claim 21, further comprising desorbing the target analyte from the MIP layer, wherein desorbing the target analyte from the MIP layer comprises heating the at least one working electrode to desorb the target analyte from the MIP layer, wherein the electrochemical biosensor comprises a microheater integrated on the electrically insulative substrate.

25. The method of claim 24, wherein desorbing the target analyte from the MIP layer comprises applying the electrochemical potential to the at least one working electrode to cause the MIP layer to swell.

26. The method of claim 24, wherein desorbing the target analyte from the MIP layer comprises applying the electrochemical potential to the at least one working electrode to move the target analyte using an electric field.
